(19) United States
(12) Patent Application Publication
(10) Pub. No.: US 2024/0366105 A1
(43) Pub. Date: Nov. 7, 2024

(54) INNER EAR ACCESS DETERMINATION

(71) Applicant: COCHLEAR LIMITED, Macquarie University, NSW (AU)

(72) Inventors: Wolfram Frederik DUECK, Macquarie University (AU); Daniel SMYTH, Macquarie University (AU)

(21) Appl. No.: 18/686,044

(22) PCT Filed: Aug. 26, 2022
(86) PCT No.: PCT/IB2022/057989

§ 371 Date: Feb. 23, 2024

Related U.S. Application Data
(60) Provisional application No. 63/237,505, filed on Aug. 26, 2021.

(51) Int. Cl. A61B 5/06; A61B 5/00

(52) U.S. Cl. CPC A61B 5/068; A61B 5/6847

(57) ABSTRACT

A method including entering a fluid containing cavity in a human with an artificial device, verifying that a portion of the artificial device has entered the fluid containing cavity based on an electrical phenomenon indicative of a sensor component supported by and/or part of the artificial device being located in the fluid containing cavity.

### CROSS-REFERENCE TO RELATED APPLICATIONS

This application claims priority to U.S. Provisional Application No. 63/237,505, entitled INNER EAR ACCESS DETERMINATION, filed on Aug. 26, 2021, naming Wolfram Frederik DUECK of Hannover, Germany as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a method, comprising entering a fluid containing cavity in a human with an artificial device and verifying that a portion of the artificial device has entered the fluid containing cavity based on an electrical phenomenon indicative of a sensor component supported by and/or part of the artificial device being located in the fluid containing cavity.

In an exemplary embodiment there is an apparatus, comprising a therapeutic substance delivery device including a therapeutic substance delivery lumen and an at least a relevant environment exposed sensor component of an electrical phenomenon sensor.

In an exemplary embodiment, there is non-transitory computer-readable media having recorded thereon, a computer program for executing at least a portion of a method, the computer program including code for analyzing input from a sensor indicative of electrical phenomena within a cochlea and code for at least one of: automatically indicating to a healthcare professional data based on data indicative of a sensor component being located in a cochlea based on an analysis executed using the code for analyzing; or automatically executing a therapeutic substance delivery action based on an analysis executed using the code for analyzing.

In an exemplary embodiment, there is a system, comprising a therapeutic substance delivery device and a computational device having the non-transitory medium, wherein the system is configured with at least one of an indicator subsystem controlled by the medium for automatically indicating to the healthcare professional the data based on the data or an automatic dispensing subsystem controlled by the medium for automatically executing the therapeutic substance delivery action.

In an exemplary embodiment, there is a method, comprising obtaining access to a middle ear of a human, moving a device sized and dimensioned to fit at least partially into a duct of a cochlea in a direction believed to be towards a duct of a cochlea from the middle ear, and artificially verifying that a distal portion of the device is statistically likely to be located in the cochlea.

In an exemplary embodiment, there is an apparatus, comprising a handle body, a termination extending from the handle body, a therapeutic substance reservoir in controllable fluid communication with the termination, wherein the apparatus is a therapeutic substance delivery device, wherein the termination is a therapeutic substance delivery lumen, at least one electrode is supported by the termination at a distal end thereof, and the electrode is part of a sensor system configured to sense impedance and/or a change in impedance and/or a phenomenon that results from a change in impedance, the impedance being between the electrode and another portion of the sensor system.

### BRIEF DESCRIPTION OF THE DRAWINGS

Embodiments are described below with reference to the attached drawings, in which:

FIG. 1 is perspective view of a human ear;

FIG. 2 is a perspective view of an exemplary cochlear stimulator implanted in accordance with an exemplary embodiment;

FIGS. 3 and 4 and 4A are schematics depicting exemplary implantable components for background purposes;

FIG. 5 is a schematic depicting an exemplary therapeutic substance delivery system for background purposes;

FIG. 6 is a schematic depicting exemplary background working ends of an embodiment that combines the embodiments of FIGS. 3 to 5.

FIGS. 7A-12A and 14 are schematics depicting exemplary embodiments according to the invention;

FIG. 13 is a schematic depicting insertion of a device into a cochlea;

FIGS. 15, 16, 17, 18 and 27 present exemplary flowcharts for exemplary methods;

FIG. 19 is a schematic depicting insertion of a device into a cochlea;

FIGS. 20-26 are schematics depicting exemplary embodiments according to the invention;

FIGS. 28 and 29 are schematics depicting insertion of a device into a cochlea; and

FIGS. 30-34 depicts diagrams showing how embodiments some devices can be utilized with robotic or machine manipulated systems.

### DETAILED DESCRIPTION

Merely for ease of description, the techniques presented herein are sometimes described herein with reference to an illustrative medical device, namely a cochlear stimulator, and in other instances, a cochlear implant. However, it is to be appreciated that the techniques presented herein may also be used with a variety of other medical devices that, while providing a wide range of therapeutic benefits to recipients, patients, or other users, may benefit from setting changes based on the location of the medical device, including methods that are used in combination with such. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. Some embodiments include the utilization of the teachings herein to treat an inner ear of a recipient that has and/or utilizes one or more of these devices. The techniques presented herein may also be used with vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc. In further embodiments, the techniques presented herein may be used with air purifiers or air sensors (e.g., automatically adjust depending on environment), hospital beds, identification (ID) badges/bands, or other hospital equipment or instruments.

The teachings detailed herein can be implemented in sensory prostheses, such as hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. Moreover, with respect to any teachings herein, such corresponds to a disclosure of utilizing those teachings with all of or parts of a cochlear implant, cochlear stimulator, a bone conduction device (active and passive transcutaneous bone conduction devices, and percutaneous bone conduction devices) and a middle ear implant, providing that the art enables such, unless otherwise noted. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

Thus, merely for ease of description, the first illustrative medical device is a hearing prosthesis. Any techniques presented herein described for one type of hearing prosthesis or any other device disclosed herein corresponds to a disclosure of another embodiment of using such teaching with another device (and/or another type of hearing device including other types of bone conduction devices (active transcutaneous and/or passive transcutaneous), middle ear auditory prostheses (particularly, the EM vibrator/actuator thereof), direct acoustic stimulators), etc. The techniques presented herein can be used with implantable/implanted microphones (where such is a transducer that receives vibrations and outputs an electrical signal (effectively, the reverse of an EM actuator), whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), and thus any disclosure herein is a disclosure of utilizing such devices with the teachings herein (and vice versa), providing that the art enables such. The teachings herein can also be used with conventional hearing devices, such as telephones and ear bud devices connected MP3 players or smart phones or other types of devices that can provide audio signal output, that use an EM transducer. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

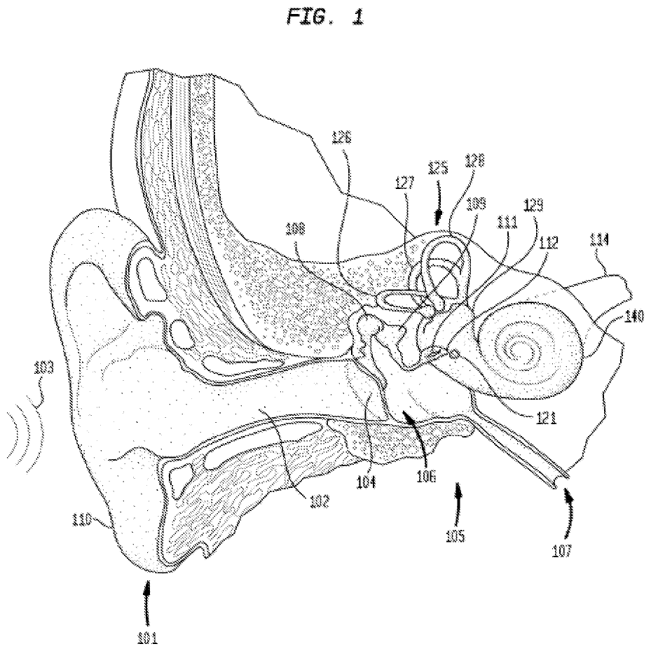

FIG. 1 is a perspective view of a human skull showing the anatomy of the human ear. As shown in FIG. 1, the human ear comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121. This vibration is coupled through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to the vibration of tympanic membrane 104. This vibration sets up waves of fluid motion of the perilymph within cochlea 140. Such fluid motion, in turn, activates hair cells (not shown) inside cochlea 140. Activation of the hair cells causes nerve impulses to be generated and transferred through the spiral ganglion cells (not shown) and auditory nerve 114 to the brain (also not shown) where they cause a hearing percept.

As shown in FIG. 1, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127, and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the orientation of the skull changes, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

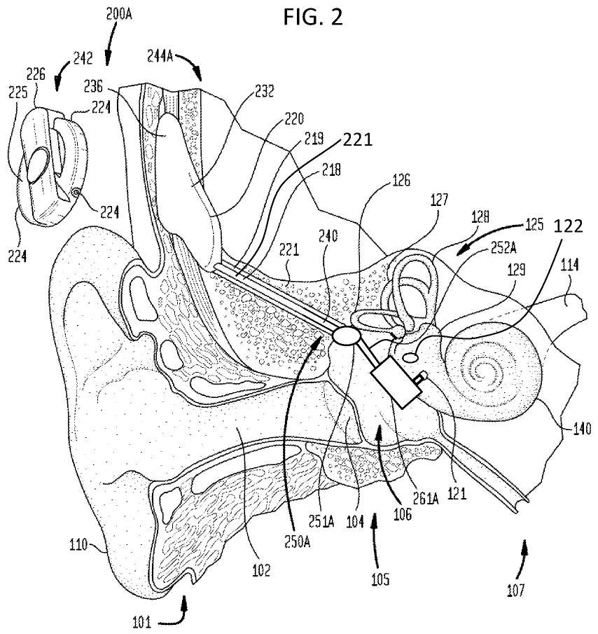

FIG. 2 is a perspective view of an exemplary cochlear stimulator 200A in accordance with some exemplary embodiments. Cochlear stimulator 200A comprises an external component 242 that is directly or indirectly attached to the body of the recipient, and an internal component 244A that is temporarily or permanently implanted in the recipient. External component 242 typically comprises two or more sound input elements, such as microphones 224 for detecting sound, a sound processing unit 226, a power source (not shown), and an external transmitter unit 225. External transmitter unit 225 comprises an external coil (not shown). Sound processing unit 226 processes the output of microphones 224 and generates encoded data signals which are provided to external transmitter unit 225. For ease of illustration, sound processing unit 226 is shown detached from the recipient.

Internal component 244A comprises an internal receiver unit 232, a stimulator unit 220, and a stimulation arrangement 250A in electrical communication with stimulator unit 220 via cable 218 extending thorough artificial passageway 219 in mastoid bone 221. Internal receiver unit 232 and stimulator unit 220 are hermetically sealed within a biocompatible housing, and are sometimes collectively referred to as a stimulator/receiver unit.

Internal receiver unit 232 comprises an internal coil (not shown), and optionally, a magnet (also not shown) fixed relative to the internal coil. The external coil transmits electrical signals (i.e., power and stimulation data) to the internal coil via a radio frequency (RF) link. The internal coil is typically a wire antenna coil comprised of multiple turns of electrically insulated platinum or gold wire. The electrical insulation of the internal coil is provided by a flexible silicone molding (not shown). In use, implantable receiver unit 232 is positioned in a recess of the temporal bone adjacent auricle 110.

In the illustrative embodiment of FIG. 2, ossicles 106 have been explanted, thus revealing oval window 122.

Stimulation arrangement 250A comprises both the distal and proximal portions of cable 218 (221 and 240), an actuator assembly 261A, an actuator mount member 251A, an actuator position arm 252A that extends from actuator mount member 251A and supports or at least holds actuator assembly 261A in place relative to the outside of the cochlea 140. In an exemplary embodiment, actuator mount member 251A is osseointegrated to mastoid bone 221, or more particularly, to the exit of artificial passageway 219 formed in mastoid bone 221.

In this embodiment, stimulation arrangement 250A is implanted and/or configured such that a portion of the actuator assembly interfaces with the round window 121, as can be seen, while it is noted that in an alternate embodiment, a portion of the actuator assembly interfaces with the oval window 122 (and both windows in some alternate embodiments).

As noted above, a sound signal is received by microphone(s) 224, processed by sound processing unit 226, and transmitted as encoded data signals to internal receiver 232. Based on these received signals, stimulator unit 220 generates drive signals which cause actuation of actuator assembly 261A.

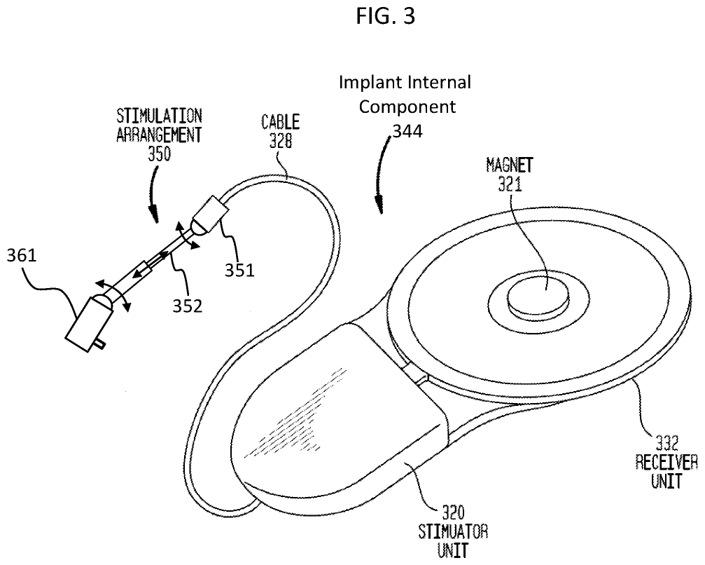

FIG. 3 is a perspective view of an exemplary internal component 344 of an implant which generally represents internal component 244A described above. Internal component 344 comprises an internal receiver unit 332, a stimulator unit 320, and a stimulation arrangement 350. As shown, receiver unit 332 comprises an internal coil (not shown), and a magnet 321 fixed relative to the internal coil. In some embodiments, internal receiver unit 332 and stimulator unit 320 are hermetically sealed within a biocompatible housing. This housing has been omitted from FIG. 3 for ease of illustration.

Stimulator unit 320 is connected to stimulation arrangement 350 via a cable 328, corresponding to cable 218 of FIG. 2. Stimulation arrangement 350 comprises an actuator assembly 361, corresponding to actuator 261A of FIG. 2, an actuator assembly mount member 351, corresponding to actuator assembly mount member 251A of FIG. 2, and an actuator assembly positioning arm 352, corresponding to the actuator assembly positioning arm 352 of FIG. 2. In an exemplary embodiment, actuator assembly mount member 351 is configured to be located in the artificial passageway 219 or adjacent thereto and fixed to the mastoid bone of the recipient. As indicated by the curved arrows of FIG. 3, the actuator assembly mount member 351 and the actuator assembly 361 are configured to enable articulation of the actuator assembly positioning arm 352 relative to those components. Further, as indicated by the straight arrow of FIG. 3, the actuation assembly positioning arm 352 is configured to telescope to provide longitudinal adjustment between the actuator assembly 361 and the actuator assembly mount member 251.

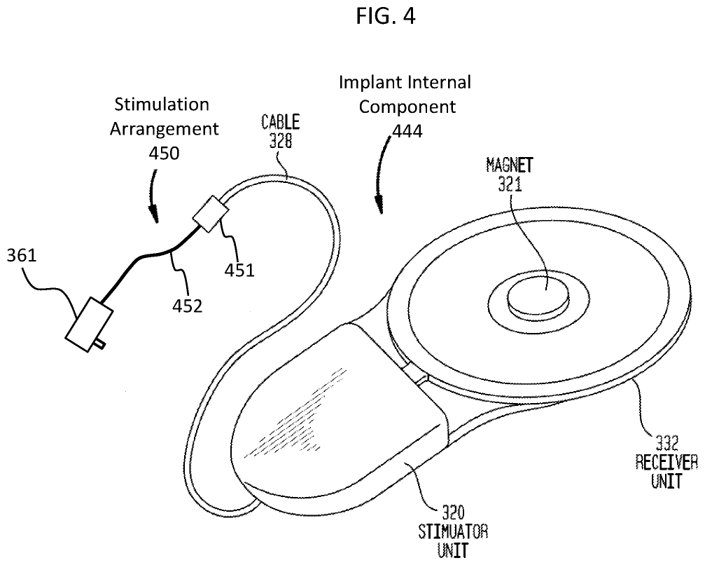

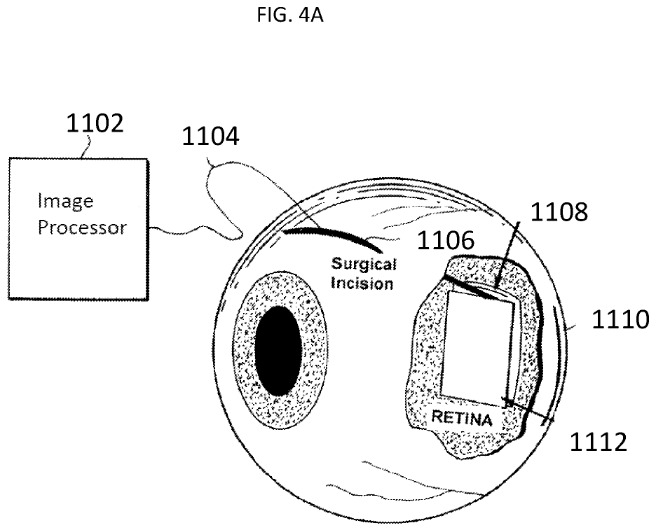

FIG. 4 is a perspective view of an exemplary internal component 444 of an implant which generally represents internal component 244A described above. Internal component 444 comprises like components corresponding to those of internal component 344.

As with internal component 344, internal component 444 is such that stimulator unit 320 is connected to stimulation arrangement 450 via a cable 328, corresponding to cable 218 of FIG. 2. However, element 451 is a coupling that instead of coupling to the articulation device detailed above in the embodiment of FIG. 3, couplies to cable 452 which is coupled to actuator assembly 361. This embodiment provides a less complicated arrangement which can have utilitarian value where the surgeon or the like is going to hand connect actuator assembly 361 directly to the exterior of the cochlea and where actuator assembly 361 will remain in place relative to the cochlea for a given period of time. The cable 452 is flexible so as to permit relative ease of movement of the actuator assembly 361 during the implantation process. The coupling 451 enables the stimulation arrangement 350 to be replaced without removing the stimulator unit 320 and/or enables the stimulator unit 320 to be removed and replaced without removing the stimulation arrangement 450.

FIG. 4A presents an exemplary embodiment of a neural prosthesis in general, and a retinal prosthesis and an environment of use thereof, in particular. In some embodiments of a retinal prosthesis, a retinal prosthesis sensor-stimulator 1108 is positioned proximate the retina 1110. In an exemplary embodiment, photons entering the eye are absorbed by a microelectronic array of the sensor-stimulator 1108 that is hybridized to a glass piece 1112 containing, for example, an embedded array of microwires. The glass can have a curved surface that conforms to the inner radius of the retina. The sensor-stimulator 108 can include a microelectronic imaging device that can be made of thin silicone containing integrated circuitry that convert the incident photons to an electronic charge.

An image processor 1102 is in signal communication with the sensor-stimulator 1108 via cable 1104 which extends through surgical incision 1106 through the eye wall (although in other embodiments, the image processor 1102 is in wireless communication with the sensor-stimulator 1108). In an exemplary embodiment, the image processor 1102 is analogous to the sound processor/signal processors of the auditory prostheses detailed herein, and in this regard, any disclosure of the latter herein corresponds to a disclosure of the former in an alternate embodiment. The image processor 1102 processes the input into the sensor-stimulator 108, and provides control signals back to the sensor-stimulator 1108 so the device can provide processed and output to the optic nerve. That said, in an alternate embodiment, the processing is executed by a component proximate to or integrated with the sensor-stimulator 1108. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 1108 captures light/images, which sensor-stimulator is implanted in the recipient. In an exemplary embodiment, there is a transcutaneous communication coil that is held against a skin of a recipient via magnetic attraction to communication with an implanted component, which implanted component provides the stimulation to evoke a sight precept. In an embodiment, the teachings herein regarding magnetic attraction are utilized in such.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

Some exemplary embodiments of the teachings detailed herein enable drug delivery to the cochlea or otherwise the delivery of a utilitarian substance to the cochlea.

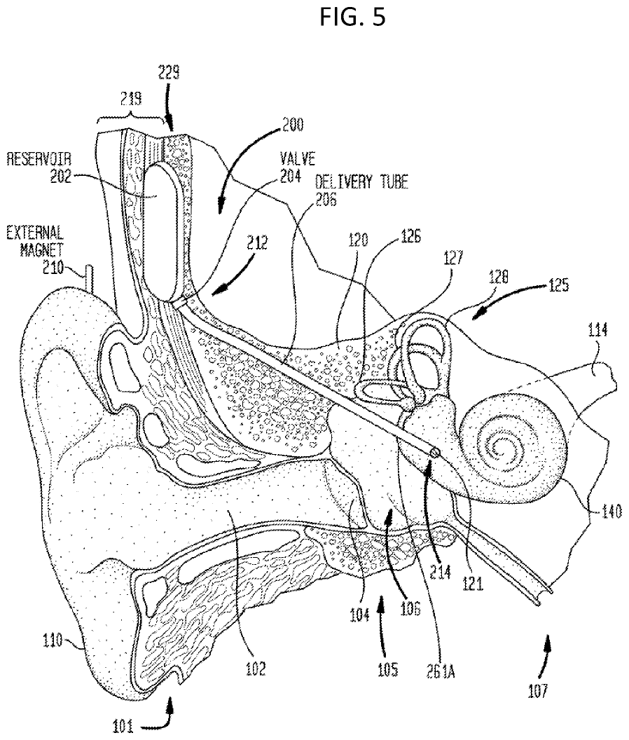

FIG. 5 depicts an exemplary drug delivery device, the details of which will be provided below. It can be utilitarian to have a prompt and/or extended delivery solution for use in the delivery of treatment substances to a target location of a recipient. In general, extended treatment substance delivery refers to the delivery of treatment substances over a period of time (e.g., continuously, periodically, etc.). The extended delivery may be activated during or after surgery and can be extended as long as is needed. The period of time may not immediately follow the initial implantation of the auditory prosthesis. Embodiments of the teachings herein can facilitate extended delivery of treatment substances, as well as facilitating prompt delivery of such substances.

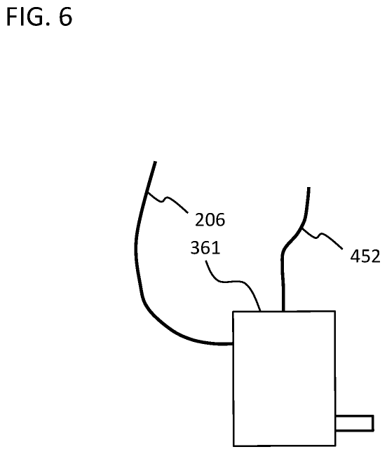

FIG. 5 illustrates an implantable delivery system 200 having an actuation mechanism, which can be modified as will be detailed below in some embodiments. However, it is noted that the delivery system 200 can also or instead have an active actuation system, again which can be modified as will be detailed below. The delivery system 200 is sometimes referred to herein as an inner ear delivery system because it is configured to deliver treatment substances to the recipient's inner ear (e.g., the target location is the interior of the recipient's cochlea 140). It is also noted that in some implementations of a modified arrangement of FIG. 5, as will be described below, the actuation mechanism enables movement of therapeutic substance to another device that in turn has an active actuation mechanism (e.g., element 361 of FIG. 6A, additional details of which are described below), where the latter is used to actually transport the therapeutic substance into a cochlea (the former is used to get the substances to the latter).

Delivery system 200 of FIG. 5 comprises a reservoir 202, a valve 204, and a delivery tube 206, in addition to some additional components, as will be described below. For ease of illustration, the delivery system 200 is shown separate from any implantable auditory prostheses. Additionally, the delivery system 200 can include, or operate with, an external magnet 210, which is separate from or part of the implantable auditory prostheses, for purposes of, e.g., controlling operation of valve 204.

The delivery tube 206 includes a proximal end 212 and a distal end 214. The proximal end 212 of the delivery tube 206 is fluidically coupled to the reservoir 202 via the valve 204.

FIG. 5, as shown, utilizes an actuation mechanism to produce a pumping action to transfer a treatment substance from the reservoir 202 to the delivery device 208 at the distal end 214 of the delivery tube 206, but again, some embodiments are modified versions of FIG. 5 that utilize active actuation.

In some implementations of FIG. 5, external force is applied on the tissue 219 adjacent to the reservoir 202 to create the external force. As will be described below, in some embodiments, an external vibratory device of a passive transcutaneous bone conduction device that vibrates to evoke a hearing percept is pressed onto the soft tissue 219 under which the reservoir 202 is located. The movement (e.g., oscillation/vibration) of the actuator causes deformations the reservoir 202 to create pumping action that propels the treatment substance out of the reservoir.

As noted, the treatment substance (sometimes herein referred to as therapeutic substance) is released from the reservoir 202 through the valve 204. The valve 204 may be a check valve (one-way valve) that allows the treatment substance to pass therethrough in one direction only.

Once the treatment substance is released through valve 204, the treatment substance flows through the delivery tube 206 to the cochlea, either directly, or indirectly via the actuator assembly 361/461. In embodiments utilizing the actuator assembly, the actuator assembly corresponds to a transfer mechanism to transfer the treatment substance from the delivery tube 206 into the cochlea 140 via the round window 121 (or oval window, or another orifice such as that established by a cochleostomy into the cochlea).

The reservoir 202 may include a notification mechanism that transmits a signal or notification indicating that the reservoir 202 is substantially empty and/or needs refilled. For example, one or more electrode contacts (not shown) may be present and become electrically connected when the reservoir is substantially empty. Electronic components associated with or connected to the reservoir 202 may accordingly transmit a signal indicating that reservoir needs filled or replaced.

As noted herein, the therapeutic delivery system of FIG. 5 can be combined with a partially or fully implanted device configured to evoke a hearing percept. By way of example only and not by way of limitation, the therapeutic delivery system of FIG. 5 can be combined with the hearing prosthesis of FIG. 3 and FIG. 4. Briefly, in an exemplary embodiment, the actuator assembly 361 can be configured so as to receive or otherwise connect to the distal end of tube 206 of the therapeutic delivery system. In an exemplary embodiment of such as depicted in FIG. 6A, where the embodiment of FIG. 4 is presented by way of example, it is to be understood that the embodiment of FIG. 6 is also applicable to the embodiment of FIG. 3.

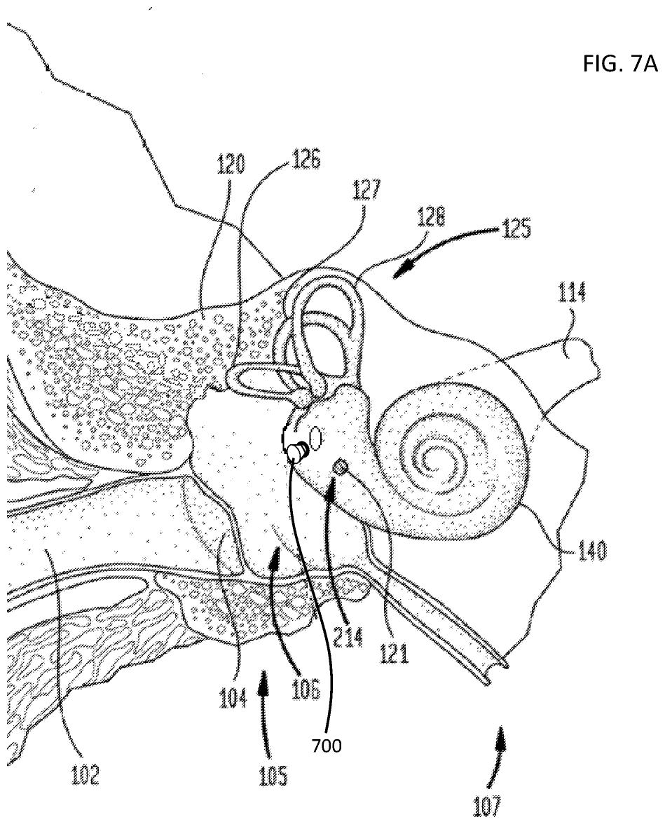

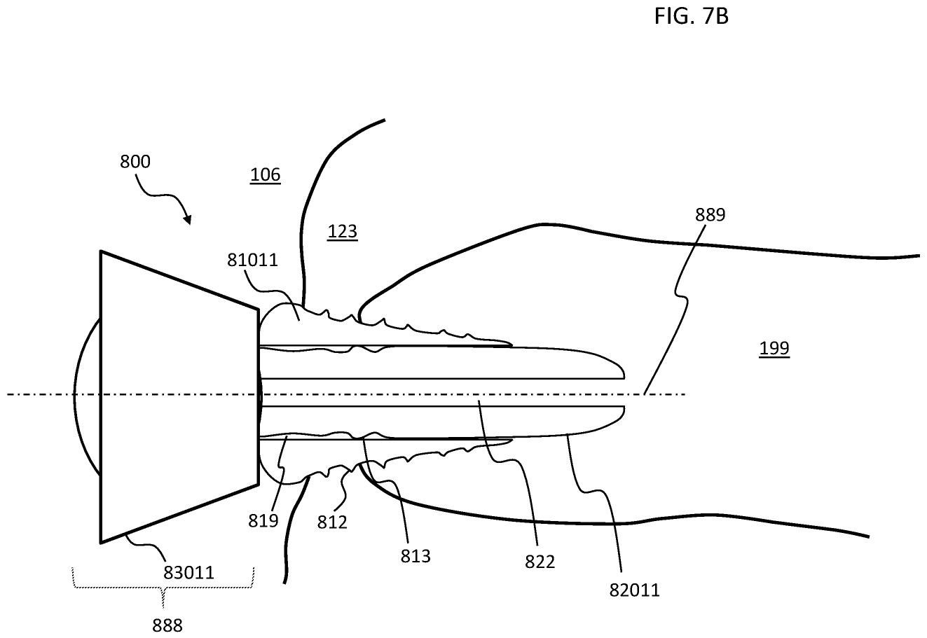

It is briefly noted by way of background that, in general, in some instances, access to the inner ear has the potential to cause damage to hearing and/or balance. In some instances, repeated access to try different therapies, or for repeated application of drugs, can be facilitated using an inner ear port. In FIGS. 7A and 7B, there is an inner ear port device 700/800. FIG. 7A depicts the visible portions of an exemplary inner ear port device 700 visible from the middle ear 106 cavity. The port device is configured to enable resealable physical access from the middle ear cavity 106 into the inner ear 199 (see FIG. 7B) through a passage through the port device 700.

FIG. 7B depicts a side view partial cross-sectional view of an exemplary embodiment of an inner ear port device 800 which can correspond to the inner ear port device 700 noted above, which extends from the middle ear cavity 106, through the bone structure 123, that divides the middle ear cavity 106 from the interior of the cochlea 199, and thus extends therethrough. The port device can extend through the promontory. The port device can extend through the barrier between the middle ear and the inner. The port device can extend through the wall of the first turn of the cochlea. The port device can extend through the bone between the round and oval window. In this embodiment, the port device 800 includes a portion that is located in or otherwise is accessible from the middle ear cavity 106. Also as seen, the port device 800 includes a portion that is located in or otherwise is in fluid communication with the cavity 199 of the cochlea, which can be one or more of the three ducts of the cochlea. In an exemplary embodiment, therapeutic substances can be transferred from a location within the cavity 106 into the cavity 199 through the port 800.

In at least some exemplary embodiments, the port device 800 is attached to the wall of the cochlea 123 at a location away from the round window and/or from the oval window. In this regard, the passage through the wall the cochlea 123 can be established via a cochleostomy through the bony structure of the cochlea 123. That said, in at least some exemplary embodiments, the port device 800 can extend through the wall of the cochlea at the location of the round window or oval window (two can be used at both locations in some embodiments), more accurately, or potentially, the former location of the round window or oval window. Thus, in some embodiments, the device is located in a cochleostomy away from a natural round window location of a human.

FIG. 7B depicts a body 81011. A passage 819 extends through that body. While in at least some exemplary embodiments, the passage 819 can include only a seal apparatus, such as by way of example, a septum, such as a self-healing septum, in this exemplary embodiment, the passage 819 has a second component, here, module 82011, located therein, which module in turn has a passage 822. Body 83011 is screwably attached to module 82011, which body forms a head of an assembly that includes module 82011 (the assembly can be considered itself a module-thus, there is a first module, body 81011, and a second module that is the assembly of head 888 and element 82011 (or, just element 82011 can be considered the second module)). In an exemplary embodiment, pulling on the head 888 pulls out the element 82011 from the passage through the body 81011.

In the embodiment of FIG. 7B, the body 81011 is configured to fix to an opening in the barrier between the middle ear in the inner ear of the human (e.g., the cochleostomy). In an exemplary embodiment, the body 81011 is configured to permanently fix to an opening in the barrier.

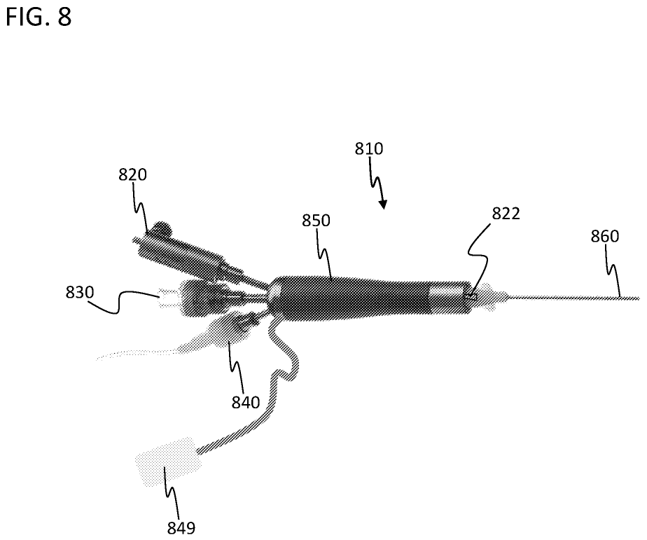

FIG. 8 presents an exemplary embodiment that is different than that disclosed in FIGS. 5-6, but which can be used with the arrangements of FIGS. 7A and 7B, but also without. In this regard, the invention of this patent application corresponds to the embodiments of FIG. 8 and the figures thereafter. Any means-plus-function claims relating to the implant as a whole correspond to the structure of FIG. 8 and/or the figures thereafter. It is noted that some exemplary embodiments of the invention utilize the structure and/or function of the teachings detailed above. And embodiments of the implants according to the invention can include one or more of the above noted structures and/or functions and/or can include methods that include one or more of the above noted method actions. This is thus related art that some aspects of the invention can utilize.

It is also noted that while the teachings detailed herein are directed towards accessing an inner ear from a middle ear (or from another part of a body), embodiments include utilizing the teachings detailed herein to extend through barriers of the human body between two cavities, which barriers are established by the bone for example, such as through the skull into the brain cavity, through an eye socket bone, through an arm bone or leg bone to reach the hollow portion thereof, through the rib cage to reach the heart or the lungs, etc.

It is also briefly noted that in some figures/embodiments, herein, the ossicles have been removed (from the figure, for example—this can correspond to the practice embodiment, or can be done in the interest of clarity). Some embodiments can be utilized with an intact ossicles, while other embodiments are utilized in a human where the ossicles of the respective middle ear cavity has been removed. To be clear, embodiments according to the teachings detailed herein are directed towards preserving hearing or otherwise treating hearing loss, and thus in some embodiments, the ossicles are present and functioning. But it is noted that the absence of the ossicles does not rule out embodiments associated with preserving hearing and/or treating hearing loss-hearing could be established via a middle ear implant and/or a bone conduction implant and/or a cochlear implant electrode array, etc. For example, embodiments of the teachings detailed herein can be utilized to preserve or otherwise prevent cilia degradation, where the ossicles have completely deteriorated to the point of not being useful from a medical standpoint-they might be there, but they do not function in a medically meaningful way for example.

It is briefly noted that by “transferred from a location,” this includes the scenario where the therapeutic substance travels through that area from a location that originates outside of the middle ear cavity 106. For example, a syringe including a substance can be located in the outer ear, and the termination can extend through the tympanic membrane, across cavity 106, and into the port device 800. Upon operating the syringe to transfer the therapeutic substance therein from the outer ear to the inner ear, the therapeutic substance passes through the middle ear 106, and thus is transferred from a location in the middle ear. This is as distinguished from a therapeutic substance that has as its origin location within the middle ear cavity 106, which could be the case with respect to a reservoir that is part of the port device, which reservoir is entirely located in the middle ear 106 (this would also include the species of the substance being transferred from the location within the middle ear 106—this would not include the species of the substance having an origination at the time of being attached or otherwise introduced to the body at a location outside the middle ear).

FIGS. 5-6 illustrate a specific example in which the round window 121 is the target location. As noted above, the round window 121 is an exemplary target location and other target locations are possible. FIGS. 5-6 also illustrate that the reservoir 202 is positioned adjacent to the outer surface 229 of the recipient's skull so that an external force may be used to propel the treatment substance from the reservoir. Embodiments can be used to access the cochlea through the round window.

While the features of FIGS. 5-6 are directed to an implantable therapeutic substance delivery system of the prior art, embodiments disclosed herein are directed towards medical tools and methods utilized by surgeons or other healthcare professionals, and, in some embodiments, by recipients or lay people (more on this below) to provide therapeutic substances to the cochlea, and, to tools and methods that provide access to the cochlea (in the first instance or on a repeated or subsequent to the first instance basis). That is, in contrast to the embodiment of FIG. 5, these teachings are directed towards a non-implantable device, although it is noted that in some embodiments, there may be an implanted component involved with the methods (but can be used with implanted components, such as the ports of FIGS. 7A and 7B). In a broad sense, the teachings below are directed to tools, not implants, in the sense that one does not implant a tool, but one may utilize a tool in conjunction with an implant. It is noted that some embodiments can use one or more of the aspects of the embodiment of FIG. 5 to enable the teachings below.

It is noted that in some exemplary embodiments, the therapeutic substance can be delivered to other portions of the ear system, such as by way of example only and not by way of limitation, the semicircular canals. Embodiments include the utilization of the techniques and/or tools etc., herein to pierce or otherwise in size the tissue of the semicircular canals, and provide therapeutic substance there to. It is noted that any disclosure herein referencing accessing and/or providing therapeutic substance to the ducts of the cochlea corresponds to an alternate disclosure of an alternate exemplary embodiment of accessing and/or providing their picks options to the semicircular canals providing that the art enables such, unless otherwise noted.

Note also that while the various teachings detailed herein are directed towards piercing or otherwise incising through the tympanic membrane and/or the round window membrane, in an exemplary embodiment, the teachings detailed herein also provide an arrangement that can enable incising through so-called false membranes as well as the true membrane.

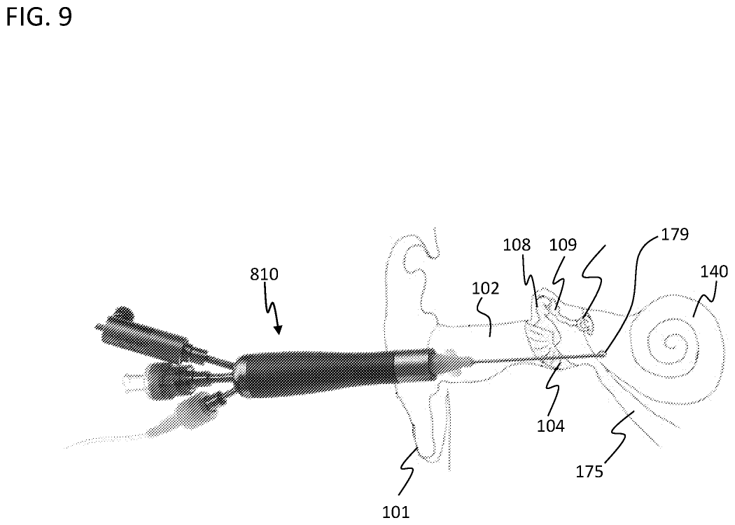

In this regard, FIG. 8 presents an exemplary embodiment of an ear system therapeutic substance delivery device 810. The ear system therapeutic substance delivery device is configured to incise through tissue to reach duct(s) of an inner ear of a human and to deliver therapeutic substance to the duct(s) through a resulting incision. More specifically, as seen in FIG. 9, the ear system therapeutic substance delivery device 810 can be seen extending into the outer ear 102/ear canal 102, with a component penetrating the tympanic membrane 104, which component extends all the way to the round window niche 179. It is briefly noted that while the embodiments disclosed above have generally identified that area labeled 179 as the round window, in reality, the round window is eclipsed at least partially by the promontory 123 or otherwise by the bone that establishes the round window niche 179. In this exemplary embodiment, the component is curved and/or angled at the end so as to bend around the round window niche and pierce the round window (the component can be flexible-more on this below). More features of this will be described below, but briefly, upon piercing the round window, a therapeutic substance can be delivered into the duct of the cochlea, and thus embodiments include methods of utilizing the ear system therapeutic substance delivery device 810 to provide therapeutic substance into the ducts of the cochlea from a location outside the middle ear, and from outside the outer ear for that matter in some embodiments, through the middle ear, and into the inner ear. Briefly, in an exemplary embodiment, the therapeutic substance on a molecular basis moves from the location outside the middle ear and/or from outside the outer ear, to the inner ear, within 1 minute, within 45, 30, 25, 20, 15, 10, or 5 seconds or less. This as opposed to, for example, that which may occur utilizing diffusion or the like where the therapeutic substances simply provided to the round window niche, and the therapeutic substance diffuses through the round window into the cochlea (or through an artificial membrane for that matter—some instances of such can include separating the middle ear from the inner ear with an artificial membrane, and the drug is applied to the middle ear facing side of the membrane for the drug to diffuse through the artificial membrane into the cochlea duct—this can be utilitarian as it can keep the cochlea a closed system but still allow a therapeutic substance to diffuse in faster, more controlled manner and/or allow diffusion of molecule side to large or unipolar size through the round window). It is noted that other embodiments of therapeutic substance delivery do utilize diffusion.

Returning back to FIG. 8, the ear system therapeutic substance delivery device 810 includes various components, such as, by way of example, an optional optical channel 820 which enables data indicative of an image inside the ear system to be conveyed to a location outside the ear system. And thus, in an embodiment, device 810 can be an ear system endoscope. Herein, that phrase may be utilized to refer to device 810. It is to be noted that device 810 need not be an endoscope, and that reference to such as an endoscope does not mean that it does not have therapeutic substance delivery capabilities. Conversely, not all endoscopes need to have the therapeutic substance delivery capabilities. Some embodiments do not.

Embodiments often focus on entering the cochlea through the round window. It is noted that other embodiments can enter the cochlea through other ways, such as through the oval window, or through a cochleostomy through the promontory. Accordingly, the teachings detailed herein are applicable to any viable way of entering the cochlea detailed herein or otherwise and embodiments include executing the teachings detailed herein with such viable ways.

It is noted that embodiments include devices that do not have the optical features of the device of FIG. 8. Some embodiments included executing the various methods herein without being able to see an area at/proximate the boundary of the cochlea (or whatever cavity is being accessed). In some embodiments, the methods herein are executed blindly with respect to the boundary through which the device is attempted to pass.

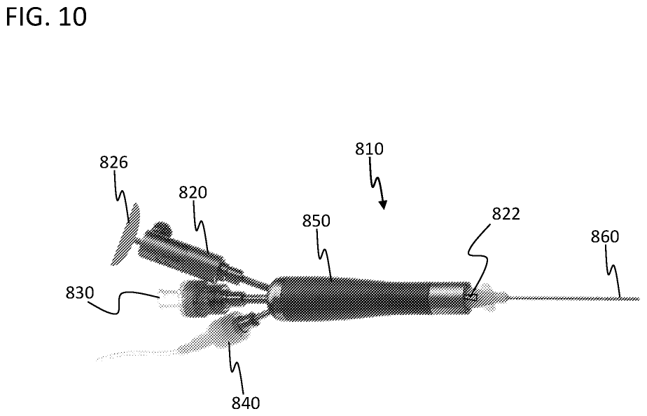

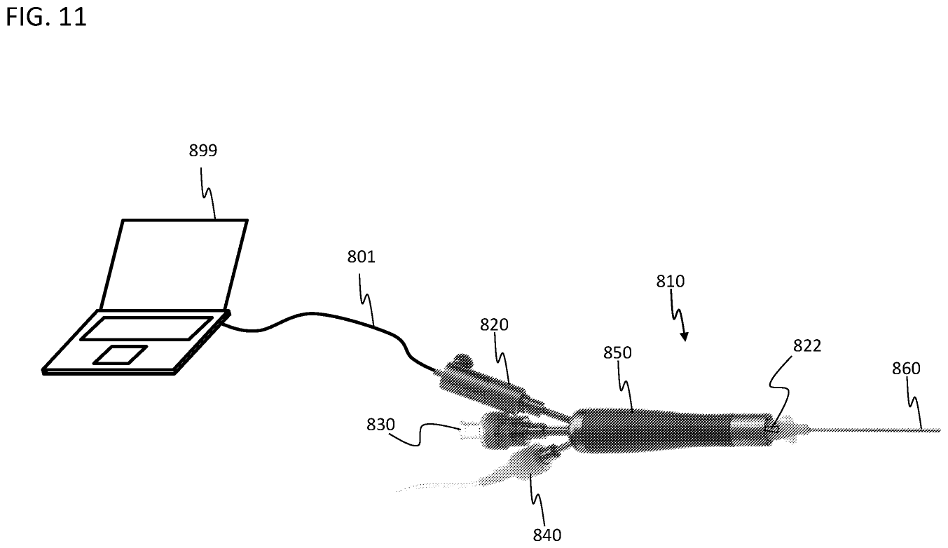

The imaging arrangement of device 810 can be based on fiber optics or wired communication. Corollary to this is that there is a camera 822 or some other light capture arrangement (a purely optical device can be used, which may magnify light captured at the working end) that is part of the ear system therapeutic substance delivery device, that is in electrical communication or light communication with the optical channel 820. The optical channel 820 can be in communication with a display 826 (see FIG. 10) or some other image conveying device, such as a lens (again, a purely optical system can be used, akin to the output of a traditional microscope, for example). Alternatively, in or in addition to this, the optical channel 820 can be configured to provide the data indicative of an image via a cable 801 (or a wireless link, such as a Blue Tooth link) to a “remote” monitor or the like positioned away from the ear system endoscope 810, such as a monitor of a laptop 899 (See FIG. 11) or a desktop computer, which could be located in the same room in which the ear system endoscope is being utilized to reach the cochlea. Accordingly, it can be seen that in some scenarios of use the surgeon or other healthcare professional or whoever is utilizing the ear system therapeutic substance delivery device guides the device through the ear system by looking at the device/looking in the direction of the side of the human's head/looking at the outer ear, while in some other embodiments, the guiding is executed while the user is looking at a computer screen and thus looking in a direction away from the human's head/outer ear, etc.

Again referring back to FIG. 8, the ear system therapeutic substance delivery device 810 further includes a surgical tool port 830. This port can be configured to receive a needle and/or a drill and/or a laser generator and/or output and/or or a micro tweezers or can just be a general port to which a therapeutic substance reservoir or a therapeutic substance supply line can be attached to deliver therapeutic substance to the inner ear. Furthermore, in an example, the port can receive a biopsy tool and/or blades and/or forceps and/or microneedles (microneedle array/assembly), catheters, etc. Moreover, by way of example only and not by way of limitation, in an exemplary embodiment, tools that are configured to take a sample of a fluid or a liquid within the body, such as perilymph within the cochlea and/or the fluid within the semicircular canals, example, can be extended through the aforementioned port(s).

But note that in some embodiments, the device 810 can contain a reservoir and/or a container of therapeutic substance, and can deliver such via a termination 860 (more on this below) irrespective of the presence or absence of the port 830. That said, a container of therapeutic substance can be attached to the port 830, and conducted in a controlled manner to the termination 860.

An exemplary embodiment includes a steerable tip ear system therapeutic substance delivery device with a channel for vision and a channel for tools (e.g., a drill tip and/or a therapeutic substance delivery tool (e.g., a conduit extends through a channel), the tool bending in compliance with the tip angle. Thus, there could be 2, 3, 4 or more surgical tool ports 830.

Also, as can be seen, there is an irrigation port 840, which can be utilized to provide irrigation fluid, such as a saline liquid, to the working end of the ear system therapeutic substance delivery device, which can be used to provide irrigation of the middle and/or inner ear during use of the tool. The tympanic membrane can also be irrigated utilizing the irrigation features of the ear system endoscope in some embodiments.

As can be seen, the channel 820 and the ports 830 and 840 are supported by a body 850, which can be ergonomically designed so that a surgeon or other healthcare professional and easily grip and support the ear system endoscope with a thumb and one or more fingers of a hand, or the entire hand.

The working end of the ear system therapeutic substance delivery device 810 includes a termination 860 (referenced briefly above), which can be a tube made of metal, such as stainless steel (e.g., 316), or some other material. In an exemplary embodiment, the termination 860 can correspond to those of at least the body portion (which may or may not include the sharp end) of a syringe termination approved for use in the United States as of Jul. 20, 2021. This can be a low volume, medium volume, or high volume termination. In an exemplary embodiment, the termination is sized and dimensioned the termination can extend from the tympanic membrane 104 to the promontory (and into the promontory) and/or to the round window niche of the cochlea, when the base 850 is inserted into the ear canal and/or is located at the beginning of the ear canal (as seen in FIG. 9).

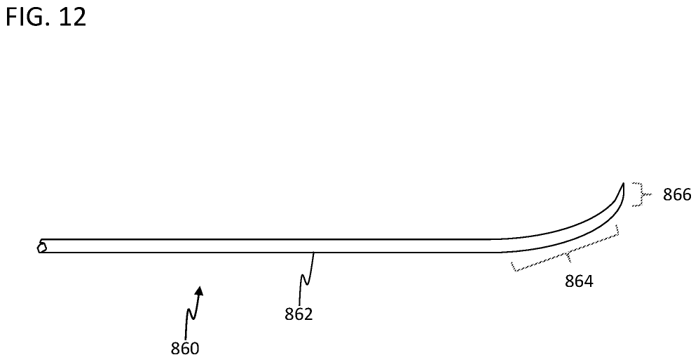

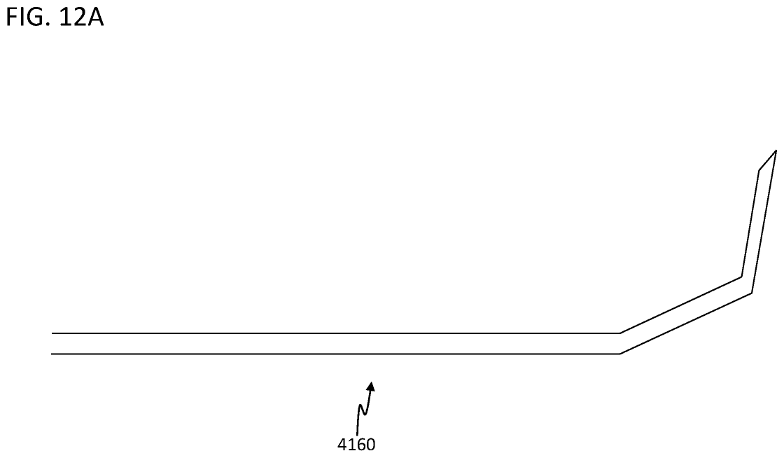

FIG. 12 depicts a portion of the termination 860. The termination 860 has a straight part and a curved part, as can be seen. In this exemplary embodiment, this can enable access to the round window through the round window niche when the termination is extended into the middle ear through the tympanic membrane. That is, the curved portion can curve around the overhang in the round window niche to reach the round window. The end of the termination 860 is configured, in at least some embodiments, in the manner that the ends of other terminations for syringes are formed (e.g., the angled cut across the tube).

In an exemplary embodiment, the termination 860 can be a rigid structure, while in other embodiments, the termination can be flexible, or at least a portion can be flexible. In an exemplary embodiment, the termination can be an assembly or a compilation that has a rigid part 862 and a flexible part 864. In this exemplary embodiment, the overall termination has a sufficiently rigid structure that will enable the termination to pierce the tympanic membrane, and then upon reaching the round window niche, the flexible portion will flex upon interaction with the tissue of the round window niche so that it can be driven to the round window and thus pushed through the round window so as to establish communication from outside the outer ear to inside the cochlea.

Note that while in some embodiments, the component that interacts with the round window can often be the conduit as disclosed herein, which conduit extends out of the termination. In other embodiments, it is the termination that interacts with the round window. Indeed, as will be detailed below, in some embodiments, there is no separate conduit. Moreover, in an exemplary embodiment, as noted herein, the termination can have varying diameters, such as a smaller outer diameter so as to accommodate the area within the round window niche, etc., while the larger diameter will be utilized over the remainder of the tool to provide structural support in a manner consistent with the traditional use of a termination of an endoscope.

In an exemplary embodiment, the flexible and/or bendable portions can be controlled via a guidewire and/or a guidewire and spring element assembly. In an exemplary embodiment, a stiffening element or the like such as a stylet can be located in or adjacent to the termination 860. By controlling the location of the stylet, such as by removing the stylet, the termination can then be caused to bend or otherwise flex. By way of example only and not by way of limitation, in an exemplary embodiment the termination has a memory, and the stylet or the like resists that memory so as to make the termination to be straight or to be curved for example. Upon removal of the stylet and/or guidewire or what have you, the termination will return to its relaxed state. Conversely, upon insertion of the guidewire and/or stylet etc., the termination will be forced away from its relaxed state.

In some alternate embodiments, instead of portions of the termination (and/or conduits disclosed below (which can be microlumens) that bend or flex, articulating joints can be utilized. FIG. 12A presents an exemplary termination 4160 where there are two separate joints as shown. In an exemplary embodiment, there can be one, two, three, four, five, six, seven, eight, or nine or more joints, or any value or range of values therebetween.

In an exemplary embodiment, there can be one, two, three, four, five, six, seven, eight, or nine or more sections of bending spaced by sections of non-bending, or any value or range of values therebetween.

While many of the embodiments disclosed herein relate to terminations and/or conduits and/or scopes, etc., having constant outer diameters, in alternative embodiments, the diameters can be varied over the length of the pertinent component.

In an exemplary embodiment, the angle(s) of the bend/flexure can be controlled. In an exemplary embodiment, the termination can be controlled or otherwise be flexed over a range of angles, measured in a plane, of at least 5, 10, 15, 20, 25, 30, 35, 40, 45, 50, 55, 60, 65, 70, 75, 80, 85, 90, 95, 100, 105, 110, 115, 120, 125, or 130 degrees, or any value or range of values therebetween in 1° increments.

In an exemplary embodiment, the termination can be a composite structure and/or an amalgamation different materials with one portion made out of a rigid structure and/or a rigid material and/or a material or structure that maintains rigidity through the process and another portion made out of the flexible material and/or has a structure that is flexible. In an exemplary embodiment, nitinol or some other material can be used, which maintains a geometry during a certain time and/or under certain circumstances (such as, for example, temperature), and then deformed. Thus, in an exemplary embodiment, section 864 can be made out of nitinol or other memory shape material, and the nitinol/termination 860 can be placed into a liquid ice and/or dry ice slurry, where section 864 achieves a relatively straight or a straight geometry, and then, after insertion through the tympanic membrane and entering the round window niche, as the termination warms (which could be a result of running warm irrigation fluid through the lumen of the termination and/or as a result of electrical current being run through a separate heating element (resistor) in the termination/next to the termination and/or via a conductive path from a remote heating device to the part that is to be heated, etc.) or run directly through nitinol to heat such up), section 864 bends around the top of the round window niche, such that the end/point 866 of the termination 860 is now aligned or otherwise pointed towards the round window. Further insertion of the ear system endoscope into the ear now results in the tip 866 of the termination being driven into and thus through the round window niche, and then into the duct of the cochlea, where the therapeutic substance can then be transferred through the termination and into the duct, and thus into the cochlea.

In an exemplary embodiment, the termination is a 27 gauge termination.

In an exemplary embodiment, an inner diameter of the lumen of the termination and/or of the various conduits detailed herein is less than or equal to 500, 450, 400, 350, 300, 250, 200, 175, 150, 140, 130, 120, 110, 100, 90, 80, 70, 65, 60, 55, 50, 45, 40, 35, 30, 25, 20, 15, or 10 micrometers, or any value or range of values therebetween in 1 μm increments.

In an exemplary embodiment, the outer diameter of the termination and/or the outer diameter of the various conduits and/or optical cable and/or the drill bits detailed herein is less than or equal to 1000, 900, 800, 700, 600 500, 450, 400, 350, 300, 250, 200, 175, 150, 140, 130, 120, 110, 100, 90, 80, 70, 65, 60, 55, 50, 45, or 40 micrometers, or any value or range of values therebetween in 1 μm increments. In an exemplary embodiment, the aforementioned values are associated with the tip of the tool that delivers the actual payload. Still, it is noted that in at least some exemplary embodiments, the terminations of the endoscopes can be somewhat larger, such as by way of example, having an outer diameter of less than or equal to 5, 4.5, 4, the 3.5, 3.25, 3, 2.75, 2.5, 2.25, or 2 mm point, or any value or range of values therebetween in 0.01 mm increments.

While the embodiments detailed herein are for the most part directed towards the utilization of the tools disclosed herein to access the cochlea for the purposes of providing a therapeutic substance therein, some embodiments are directed toward utilizing the teachings detailed herein to implant a cochlear implant electrode array into the cochlea. Accordingly, in an exemplary embodiment, there are methods that utilize one or more or all of the teachings detailed herein, modified albeit in a utilitarian manner, to implant a cochlear implant electrode array into the cochlea. It is also noted that in an exemplary embodiment, there are methods that utilize one or more or all of the teachings detailed herein, modified albeit any of the solitary manner, to implant other types of stimulators, such as stimulators for balance and/or to address tinnitus. Thus, any disclosure herein of utilizing the teachings herein with respect to a therapeutic substance corresponds to an alternate disclosure of utilizing those teachings to implant a cochlear implant electrode array and/or a balance implant and/or a tinnitus treatment device and/or an intracochlear mechanical stimulator. By way of example only and not by way of limitation, in an exemplary embodiment, element 300 of FIGS. 4A to 4E can correspond to the terminations and/or the other conduits or the like disclosed herein.

It is noted that in at least some exemplary embodiments, the teachings detailed herein are directed to specific devices that have a limited utility or use. For example, the ear system therapeutic substance delivery device is a device utilized for the ear system, and not for anything else. In this regard, in an exemplary embodiment, the various teachings detailed herein can be directed to FDA approved products for use in a given field for a given purpose and for nothing else.

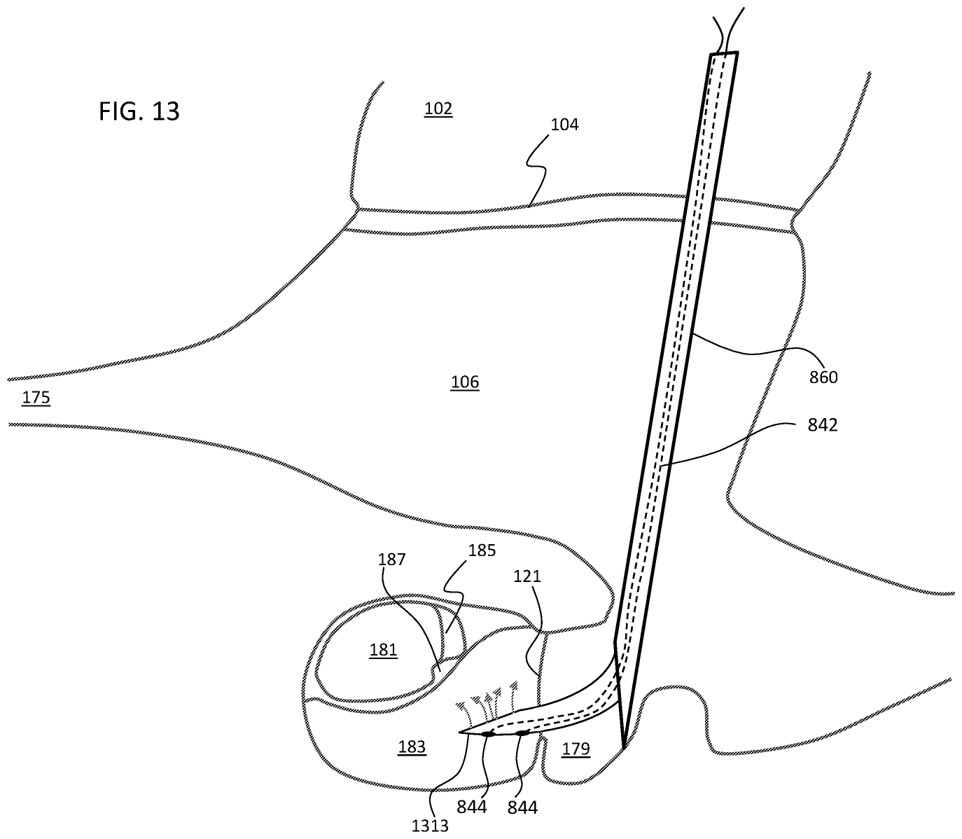

FIG. 13 represents a conceptual example of how the inner ducts of the cochlea can be accessed utilizing an exemplary ear system endoscope/ear system therapeutic substance delivery device. It is noted that in an alternate embodiment, the cochlea can be accessed through the port of FIG. 7A or 7B, etc. Briefly, shown is a cross-section of the outer middle and inner ear, where the scala tympani 183 is shown below the scala vestibule 181. For frame of reference, also shown is the scala media 185 and the organ of Corti 187. Here, the ear system endoscope includes a secondary fluid conduit 1313 that is movable relative to the termination 860 in general, and can move inside the lumen of the termination 860 in particular. In this exemplary embodiment, the termination 860 is utilized to puncture through the tympanic membrane 1040 as shown. The termination 860 is then extended downward into the round window niche 179. In this exemplary embodiment, the tip of the termination 860 is utilized to provide a “brace” on the bone so as to steady the termination 860 in general, and the distal portion thereof in particular. The sharpness of the tip can be utilized to slightly “dig” into the bone. In some embodiments, this is not necessarily the case. For example, the ear system endoscope can be positioned so that the tip of the termination 860 is held proud relative to the surface of the bone.

Upon puncturing the round window 121, the therapeutic substance can be delivered under pressure or by whatever transport regime that can have utilitarian value through the inner lumen of the conduit 1313, and thus into the scala tympani 183 as shown, where the therapeutic substance delivery is represented by the arrows extending from the chamfer of the conduit 1313.

It is noted that while the embodiment of FIG. 13 is presented with respect to the conduit 1313 within the lumen of the termination 860, the general concept of FIG. 13 also can be utilized to represent the utilization of the flexible termination 860 as detailed above and/or other arrangements (e.g., where the end of the termination is curved).

Embodiments can be directed to steerable conduits 1313 and/or steerable terminations 860.

In an exemplary embodiment, conduit 1313 can be used to pierce the self-healing septum of the port of FIGS. 7A/7B, and thus reach the interior of the cochlea, or can extend through the port by removing the cap, etc., of the port. Any regime of accessing the cochlea and/or the semi-circular canals can be used in some embodiments with the teachings herein.

In an exemplary embodiment, the device includes wires 842 or otherwise an electrically conductive material configured to conduct electrical current for the purposes of conducting an electrical signal (in alternate embodiments, this could be fiber optics and/or the body of the conduit could be used as a ground/return). In an exemplary embodiment, the electrically conductive material can be lead wires (and can extend in the conduit, and can be insulated to electrically isolate the conductive material from the therapeutic substance in the conduit). In an exemplary embodiment, the device includes electrodes 844 that are connected to the lead wires 842. In an exemplary embodiment, the electrodes and/or the wires can be supported by/be part of the termination 860, such as where the termination is used to enter the cochlea ducts.

It is noted that in an alternate embodiment, there are no definitive separate electrodes 844 that are distinguishable from the leads. Instead, the electrodes could be the bare wires that extend out of the body of the conduit or otherwise “breach” the outer surface thereof (or the outer surface of the termination in the alternate embodiment). In this exemplary embodiment, the electrodes 844, whether they be distinct separate electrodes from leads or the ends of the leads, are electrically conductively exposed to the fluid within the cavity 183. In an exemplary embodiment, a potential between the electrodes and/or impedance (as used herein, impedance refers to electrical impedance unless otherwise noted) between the electrodes can be measured to ascertain a latent variable that can be utilized to evaluate the perilymph within the cavity 183 for example, or otherwise to determine that the distal end of the conduit 1313 is in contact with the perilymph (and thus in the cavity 183). In some embodiments, this could be used to determine the presence or absence of perilymph within the cavity 183 or otherwise a qualitative feature of the perilymph within the cavity 183.

In an exemplary embodiment, the device 810 includes electronics package 849. Electronics package is depicted as being connected to the remainder of the device via an electrical cable. In an exemplary embodiment, this can be utilized to provide current flow across the two electrodes 844. In an exemplary embodiment, element 849 can be a computer chip and/or even a microprocessor. In an exemplary embodiment, the electronics package is integrated with the remainder of the device (the cable is not present-fixed leads are used).

In an exemplary embodiment, the electronics can be part of a sensor that is configured to sense one or more phenomenon, such as electrical phenomena and/or chemical phenomena and/or physical phenomena (e.g., movement of the perilymph/density of the perilymph/viscosity of perilymph, etc.). In an exemplary embodiment, the electronics package can correspond to the computer 899 detailed above. Any feature detailed herein associated with the computer 899 can correspond to that which is possessed by the electronics package 849, and vice versa, unless otherwise noted providing that the art enable such.

In an exemplary embodiment, any disclosure herein of the electrodes 844 corresponds to an alternate disclosure where elements 844 are instead and/or in addition to electrodes, another type of sensor or any of the sensors detailed herein providing that the art enables such unless otherwise noted.

In some embodiments, simple electrodes and/or devices that measure/monitor impedance and/or monitor/measure electrical activity are utilized in at least some exemplary embodiments of the devices that utilize sensors. In some embodiments, the sensors are biosensors for detecting specific proteins or other biomolecules, or cells. In some embodiments, the sensors are configured to monitor or detect or otherwise evaluate the presence and/or absence of glucose (including determining levels), bacteria, small molecule sensors for metals, for example, or specific small molecule sensors for drugs/artificial substances in the cochlea, or the presence and concentration of specific ions such as sodium and potassium, and some sensors can be MIPS (molecularly imprinted polymer sensors).

Embodiments thus enable method actions, and include method actions of utilizing perilymph detection in scenarios where a needle of a therapeutic substance delivery device, for example, penetrates the round window membrane (or the other membrane, or through the bone of the promontory) to deposit a therapeutic substance into perilymph in the cochlea. In this scenario, the perilymph detection provides objective feedback to the practitioner or person executing the method that the working end of the needle (lumen, conduit, etc.) has indeed penetrated the round window membrane (or the other membrane, or through the bone of the promontory, or whatever opening is used, such as a cochleostomy) and the needle is inside the inner ear. In some embodiments in addition to confirming the termination is inside the inner ear the feedback also confirms a desired or utilitarian distance or length of the termination extending into the inner ear. In some embodiments, this can be a trigger for automated or therapeutic substance administration from the needle. In some embodiments, this can be an indication to the practitioner that he or she can begin transferring the therapeutic substance.

Some embodiments include impedance reduction due to contact with perilymph. An embodiment can include using standard platinum electrodes. In an exemplary embodiment, the electronics package includes processors and/or chips or otherwise circuitry to implement impedance measurements according to those commercially available for use with cochlear implants, such as CustomSound™ from Cochlear Limited, where a positive voltage is applied for a first time period, and then after a waiting period (e.g., about ¼^{th} of the first time period), an opposite negative voltage is applied for a second time period equal to or about equal to the first time period). Recording times are at trailing edges of the positive pulse-phase of such.

Thus, in view of the above, there is an apparatus, comprising a therapeutic substance delivery device including a therapeutic substance delivery lumen (e.g., the lumen established by conduit 1313, or by termination 860, etc.), and an at least a relevant environment exposed sensor component (e.g., electrodes 844, as opposed to the leads, which are not exposed to the environment, at least do to the shielding/insulation) of an electrical phenomenon sensor (where the sensor can include the electrodes, the leads 842, and the electronics package 849, where the leads 842 are in electrical communication with the cable and thus the electronics package 849). As detailed above, the electrical phenomenon sensor is configured to sense impedance and/or a change in impedance and/or a phenomenon that results from a change of impedance. The impedance being between the at least a relevant environment exposed sensor component and another component of the sensor. Here, this could be the impedance between the two electrodes 844. This can also be the impedance between electrode, and a common ground, which common ground could be the body of the conduit, where there is insulation between the electrode and the body of the conduit. Still, there can be utilitarian value with respect to utilizing two electrodes having a precise and known design spaced precisely away from one another and having precisely understood electrical characteristics so that the measurements obtained from utilization of the sensor can be analyzed with heightened specificity. For example, the more that the test components are controlled and otherwise the more that is known about the test components, the narrower the range of data from the sensor with respect to the former, and the more precise the data can be analyzed with respect to the latter. This because, for example, the tolerancing and/or variation induced by the tightened control of the working elements will be limited relative to that which would otherwise be the case.

Thus, as can be seen, in at least some exemplary embodiments, the at least a sensor component of the electrical phenomenon sensor includes at least two electrodes supported by structure establishing the therapeutic substance delivery lumen (e.g., the conduit 1313). In an exemplary embodiment, the electrodes will be electrically insulated from the conduit. In an exemplary embodiment, the conduit is made of material that is nonconductive or otherwise relatively low conductive relative to the perilymph and/or the material of the electrodes.

In an exemplary embodiment, the insulation and/or material of the conduit can be a material that is at least 10, 15, 20, 30, 40, 50, 75, 100, 150, 200, 300, 400, 500, or 1000 times or more, or any value or range of values therebetween in 1 increment more resistive to the conduction of electricity than the electrodes and/or the perilymph (statistically average perilymph (mean, median and/or mode)), in a 5 volt 10, 25, 50, 75, or 100 milliamp environment.

Concomitant with the embodiments detailed above, in an exemplary embodiment, the therapeutic substance delivery device is configured to extend through a round window of a human cochlea, and in some embodiments, without permanently damaging the round window. In an exemplary embodiment, the round window is a round window corresponding to a human factors engineering 50 percentile male and/or female of the age 30, 35, 40, 45, 50, 5, 60, 65, 70, 75, or 80 years of age born in the United States of America and/or the nation states of the European Union as of Jul. 20, 2021, and/or the Commonwealth of Australia and/or the People's Republic of China. It is noted that in some embodiments, the teachings herein can be used with a catheter, for example, extending through the round or oval window membranes which remain there permanently.

In an exemplary embodiment, perilymph specific impedance changes can be utilized. Here, for example, the electrodes, such as platinum electrodes, could be coated and impedance measures can be executed. For example, a special electrode with, by way of example, MIP (Molecular Imprinted Polymers) coating, ion selective coatings, or some other coating, which changes impedance when brought in contact with perilymph and/or a specific substance and/or a specific protein or ionic composition only present in perilymph, can be utilized. Such embodiments can have utilitarian value with respect to obtaining a delivery device where the impedance changes and/or the electrical phenomenon changes (the teachings detailed herein are not limited to impedance, but any phenomenon that can have utilitarian value with respect to establishing that the working end of the therapeutic substance delivery device is in the cochlea, or otherwise in contact with perilymph) occur, as a statistical matter, only when the electrodes or other environmentally exposed elements are in contact with the perilymph. If the perilymph “dissolves” or otherwise changes a state of an existing coating that shields the electrode(s) for example, and other body fluids to not do so or otherwise do it in a different manner, the statistical likelihood that the delivery device is in contact with perilymph is high. In an exemplary embodiment, the coating is a highly resistive material, or otherwise establishes higher impedance between the electrodes in a scenario where in the absence of the coatings, there would be low impedance.

Embodiments utilizing MIP coatings, etc., can be configured so that specific findings of a protein to the coating results in a biomarker that can be utilized to implement some of the teachings detailed herein, such as utilizing data indicative of the location of the distal portion of the therapeutic substance delivery device, where such is in contact with perilymph for example which has that given protein. In an exemplary embodiment, a color change can occur. As will be described in greater detail below, the sensors can be wavelength sensitive devices, and thus could detect a change in the wavelength. In some embodiments, the coatings are single-use coatings. Accordingly, embodiments include enabling a healthcare professional or even a non-healthcare professional, such as a person undergoing the treatment, to reapply a coating after and/or before each use, the applied coating resulting in the device regaining the functionality associated with the coating prior to use. Embodiments thus include devices and/or systems that enable such.

Thus, embodiments can use biosensors that are electrochemical biosensors. In an exemplary embodiment, an analyte, which can correspond to any of the fluids that would be located in any of the cavities inside a human with which the teachings detailed herein can apply, can come into contact with a biomolecule that is located on and/or is part of a transducer, or otherwise in intimate contact with the transducer. The transducer is in signal communication with an electronic circuit that is part of the sensor arrangement. The biosensor consolidates biomolecules that come into intimate contact with the transducer to yield an electrical signal equivalent to a single analyte. In an exemplary embodiment, the sensor component is a platform where chemical reactions between the analyte and the biomolecules occurs. The surface of the transducer thus has the biomolecules. Here, the transducer can transform one type of energy, such as chemical energy, into an electrical signal. The electronics process the signal, to obtain a signal that is utilitarian with respect to identifying the particular analyte at issue, or otherwise providing sufficient data to make a determination rising to the level of the specificity needed to determine the location of the distal portion of the therapeutic substance delivery device.

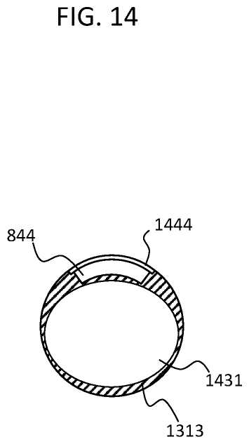

FIG. 14 presents a cross-section of a conduit 1313 showing electrode 844 coated with a coating 1444. Here, it can be seen that the lumen 1431 of the conduit 1313 is elliptical, thus providing an easier bending path up and/or down relative to the left and right owing to the relative thicknesses of the material of the conduit about the lumen (thus increasing the likelihood that the conduit will curve in the desired direction when the conduit leaves the termination, and thus had towards the round window). As can be seen, the coating 1444 extends beyond the outer periphery of the electrode 844. In this exemplary embodiment, it covers the entirety of the electrode 844 relative to the ambient environment. Note that this is still an environmentally exposed electrode. In some embodiments, the coating 1444 may extend only over part of the electrode, where the electrical current, albeit limited relative to that which would otherwise be the case in the absence of the coating, that can flow through the opening to the other electrode, where the flow of current can jumpstart or otherwise increase the rate of dissolution and/or reaction of the coating. And it is noted that while in some embodiments, all of the electrodes are coated, in other embodiments, it could be that only one electrode is coated. And while some embodiments focus on a solid coating with respect to the state of the coating at least immediately prior to entry into the cochlea, in some other embodiments, the coating could be a liquid coating. The coating could be a gel coating. Any coating that can have utilitarian value can be utilized in at least some exemplary embodiments.

In an exemplary embodiment, the coating is a coating that is utilized in view of the fact that there are protein(s) or some other substance that are statistically commonly found in perilymph (per the above noted human factors engineering cases, for example) that is not present in blood or other body tissues proximate the cochlea is the substance that will create a reaction or otherwise dissolve or change the coating, one can presume a high likelihood that the coating has indeed come into contact with perilymph when the coating reacts or otherwise dissolves to change the impedance, and thus the delivery device is positioned in a manner where there is utilitarian value to begin transporting therapeutic substance into the cochlea. In some embodiments, the absolute concentration and/or relative concentration ratio of two or more compounds such as, for example, sodium and potassium ions form the basis of the determination. That is, two or more compounds can occur in other tissue, but there is a unique ratio of the two or more in perilymph. Thus, the reaction occurs based on the ratio/absolute concentration occurring within a given range and not others. Corollary to this is that in some embodiments, the devices detailed herein are configured to determine ratios/absolute concentrations and, in some instances, automatically act/indicate based on the determined ratio (if the ratio is a certain value that falls within a predetermined range for such in perilymph, then it can be deduced by the device, using a chip or software, etc., that the sensor is in the presence of the perilymph. Note other substances may be usable (beyond sodium potassium used in the above example).

In an embodiment, the electrical phenomenon sensor is configured to measure a concentration of two or more constituents of body fluid using ion selective membrane coatings on electrode(s) of the electrical phenomenon sensor. For example, there can be one or more electrodes having coatings to measure the concentration of two or more types of constituents of a body fluid (e.g. Na+ and K+) using, for example, ion selective membrane coatings. Some embodiments include measuring the resulting current between both ion selective electrodes and a 3rd reference electrode at a given voltage. The difference in current should be the relative difference in Na+ and K+ concentration which is specific to perilymph. The difference in concentration of those two would be enough to be specific for perilymph, and thus provide an indication that the tip (given electrode at issue) is in the presence of perilymph. The absolute concentration is not needed/not used in some embodiments. Instead, the relative concentration is used, or more accurately, the changes in electrical properties resulting from the relative concentrations, etc., can be used to determine the location of the device. In this regard, in scenarios where perilymph has a unique make-up when it comes to sodium and potassium concentration, relative to other body fluids, sensors can be configured and developed to rely on this unique make-up so that the sensor will provide a signal that is unique to the electrode(s) being exposed to this unique makeup and thus provide indication of the location of the device.

And to be clear, dissolving and/or reacting are not necessarily the only ways to change the impedance. Any device, system, and/or method that can enable the teachings detailed herein can be utilized in some embodiments.

Embodiments above have mostly focused on the utilization of a single signal indicative of an electrical phenomenon between two electrodes were between electrode and a ground source for example. Embodiments can utilize two or more signals indicative of separate electrical phenomenon (the basic phenomenon can be the same, but they can be phenomenon associated with different things). By way of example only and not by way of limitation, two separate measurements can be taken utilizing three or more electrodes. By way of example only and not by way of limitation, there could be a common ground electrode located in the cochlea, in two separate hot electrodes also located in the cochlea. Separate readings can be taken based on the impedance between the common ground and one electrode, and the common ground and the other electrode. Also, the various coatings detailed herein can be utilized, where one of the two electrodes is coated with a substance that reacts to, for example, potassium, and the other of the two electrodes is coated with a substance that reacts to, for example, sodium. In an exemplary embodiment, the impedance between the respective electrodes and the ground will be different depending on the concentrations of the potassium and/or sodium, and thus the relative concentrations can be deduced based on the impedance. And note that the ground electrode may not necessarily be located in the cochlea in some embodiments. To be clear, in the aforementioned example, the two electrodes are exposed to perilymph, and the third electrode/the ground electrode, may or may not be exposed to perilymph.

Thus, it can be seen that in an exemplary embodiment, multiple readings can be obtained and compared to each other to execute at least some of the utilitarian actions detailed herein. In an exemplary embodiment, in the aforementioned example, the signals can be compared to each other, and if the data associated with the signals are not indicative of signals that would correspond to the concentrations of the substances expected to be in the cochlea, it can be determined that the electrodes at issue are not yet in the cochlea or otherwise at least not exposed to perilymph for example, and vice versa. Further, it can be seen that in some embodiments, the at least a sensor component of an electrical phenomenon sensor includes at least two electrodes supported by structure establishing the therapeutic substance delivery lumen, the at least two electrodes being independently operable to test for different physiological features within a cochlea and/or the device is configured to output at least two separate signals based on the at least two electrodes. Put another way, the device can have two or more channels (using the parlance of a cochlear implant).

The utilization of multiple readings from different configured electrodes (different coatings, for example) can be utilized to work with other unique biomarkers in this regard. The point is that embodiments are not just directed to a single signal evaluation, but also include evaluating the results of multiple signals from different sensors (2, 3, 4, 5, 6, 7, 8, 9 or 10 or more signals from different sensor components (e.g., 2 to 10 different electrodes, were there in addition could be 1 or more common electrodes).

And further, it is noted that the concept of utilizing the aforementioned coatings can have utilitarian value with respect to lessening the likelihood of a false positive reading. In this regard, the coatings shield the electrodes until the electrodes are exposed to perilymph. Depending on the material that is utilized for the coating, the electrodes would remain shielded if the electrodes were exposed to other types of body fluids that would not dissolve or react with or otherwise do not contain materials that would dissolve or otherwise react with the coating. Further by way of example, it could be that there are some body fluids outside the cochlea that could reduce the impedance between the electrodes if the fluids came into contact with the electrodes. By providing the coatings, even if the sensor components come into contact with a substance that would otherwise reduce the impedance between the electrodes, if that substance is not contain the given proteins located in the perilymph for example or otherwise does not have the chemical makeup of perilymph, the coating would not react or otherwise dissolve, etc., and thus the impedance between the electrodes would remain relatively high.

Accordingly, embodiments include shielding the electrodes, or at least the conductive parts of the electrodes until the electrodes are in the vicinity of perilymph or the like. The embodiments detailed above utilize coatings, but as will be described in further detail below, other embodiments utilize mechanical structure to achieve such shielding.

In any event, in an exemplary embodiment of the apparatus under discussion includes a sensor shield configured to unshieldably shield one or more sensor components. In an exemplary embodiment, this shield can be a chemical-reaction based shield and/or a mechanical based shield.

In an exemplary embodiment, the electrical phenomenon sensor includes at least one electrode including and/or coated with a material that is influenced by contact with perilymph, and the electrical phenomenon sensor is configured to provide a reading indicative of the electrical phenomenon sensed by the electrical phenomenon sensor that is different after the influence than before the influence.

In an exemplary embodiment, the material of the electrode and/or the material that coats the electrode is a material that is influenced by a protein of the perilymph and/or substances of the perilymph that is found only in perilymph relative to other substances that are statistically commonly found (per the above-noted human factors engineering data, for example) proximate the cochlea, such as substances found in the middle ear and/or the outer ear. In an exemplary embodiment, the proteins and/or substances are found only in perilymph relative to other substances that are statistically commonly found within 10, 15, 20, 25, 30, 35, 40, 45, 50, 55, or 60 millimeters, or any value or range of values therebetween in 1 mm increments of the inner ear and/or the ducts of the cochlea (again, by way of example, per the above noted human factors engineering data), at least with respect to locations between the inner ear and/or the ducts of the cochlea and the closest outer surface of a human's head (and thus not what is “behind” the cochlea, further in, but only in some embodiments). In an exemplary embodiment, the material of the electrode and/or coating is a material that is influence by relative and/or absolute concentrations/ratios of various chemicals. Thus, an embodiment can include using ion selective coatings on the electrodes and/or ion selective material on the electrodes.

Concomitant with the teachings above, in an exemplary embodiment, the electrical phenomenon sensor includes at least one electrode including and/or coated with a material that is influenced by contact with an artificial substance and/or a therapeutic substance, and the electrical phenomenon sensor is configured to provide a reading indicative of the electrical phenomenon sensed by the electrical phenomenon sensor that is different after the influence than before the influence. By way of example only and not by way of limitation, in an exemplary embodiment, the apparatus can be utilized to access the cochlea where previously, the therapeutic substance and/or artificial substance was delivered to the cochlea. In some scenarios where, for example, the utilization of perilymph or the materials that are normally naturally therein are not desired for use to detect and impedance change or otherwise do not enable a sufficient impedance change of a shielded electrode, the prior delivery of such substances can instead be utilized. By way of example, the coating could react to a certain type of therapeutic substance previously administered to the cochlea. The coating may not react to perilymph per se. But because the therapeutic substance is located in the cochlea, the presence thereof can be utilized to implement the teachings detailed herein. Alternatively, and/or in addition to this, the substance may not necessarily be a therapeutic substance, but instead could be a benign material/substance that is utilized for the purpose, including the sole purpose, of enabling an impedance change and/or enabling the establishment of a circuit having a readily identifiable impedance that corresponds to the impedance that would be indicative of the electrode(s) being in contact with the material/substance. That is, instead of relying on the natural substances in the cochlea to operate the sensor, embodiments can utilize human synthesized substances that are artificially present within the cochlea.

And to be clear, while some embodiments are directed towards material/substances that are artificially placed in the cochlea that will react with the aforementioned coatings, etc., in other embodiments, the material/substances or materials/substances that will provide a more accurate or otherwise more predefined electrical phenomenon reading, such as an impedance reading. By way of example, if the material/substances that are artificially located in the cochlea are such that they will result in an impedance reading that is statistically unlikely to result in a human (such as, for example, the aforementioned human factors engineering humans detailed above) that does not have this material/substance therein, a high likelihood exists that the electrodes are in contact with that material/substance. And if it is noted that the material/substance is typically (including only) located in the cochlea and not likely to be located anywhere else proximate to the cochlea, it can be determined with high likelihood that the electrodes are indeed in the cochlea. Of course, it is noted that this concept can also be applicable to natural perilymph. If the sensor is configured to provide sufficient accuracy to enable the distinguishment of an impedance reading that results from the presence of perilymph from a reading that results from other tissue and/or other body fluids or even water for example, a high likelihood can exist that the electrode is indeed in the presence of perilymph.

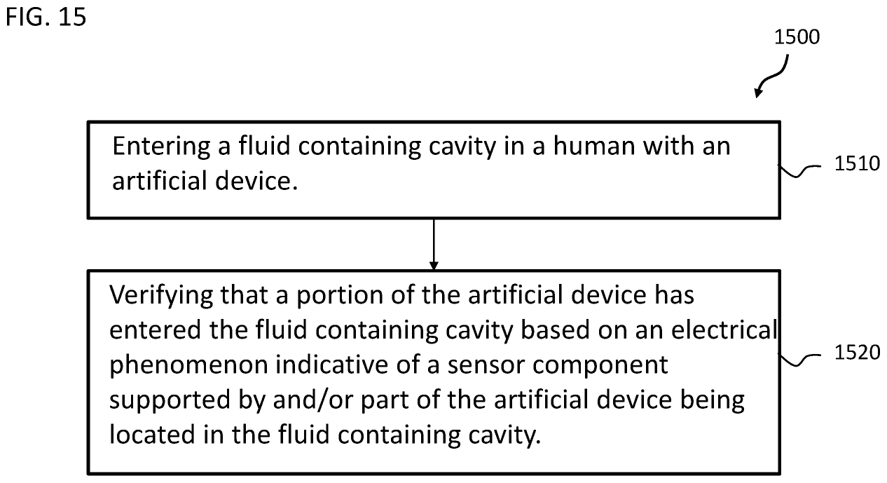

Embodiments include methods. FIG. 15 presents an exemplary flowchart for an exemplary method, method 1500, according to an exemplary embodiment. Method 1500 includes method action 1510, which includes the action of entering a fluid-containing cavity in a human (a live human) with an artificial device. This artificial device can be the conduit of the therapeutic substance delivery device and/or the termination thereof by way of example only and not by way of limitation. As will be described below, is artificial device could be a drill bit or the like. In an exemplary embodiment, the fluid containing cavity can be a cochlea. In an exemplary embodiment, the fluid containing cavity can be a duct of the cochlea of a human. In some other embodiments, the fluid containing cavity can be the semicircular canals of the inner ear. It is noted that some embodiments include accessing other containing cavities, such as by way of example only and not by way of limitation, arteries and/or veins cavities of the heart and/or lungs, etc. Method 1500 further includes method action 1520, which includes verifying that a portion of the artificial device has entered the fluid containing cavity based on an electrical phenomenon indicative of a sensor component (e.g., the electrodes 844, or a portion of the conduit 1313 that serves as a sensor component) supported by and/or part of the artificial device being located in the fluid containing cavity. Consistent with the teachings detailed above, in an exemplary embodiment, the electrical phenomenon is based on impedance. In an embodiment, this (verifying) can entail actually performing the measurements/evaluating the data. In an embodiment, this can entail receiving data from somewhere else (where the data was calculated remotely) that indicates that the artificial device has entered the cavity (which is thus verifying).

In an exemplary embodiment, the electrical phenomenon is a change in a value indicative of the electrical phenomenon. In this regard, it is noted that in some embodiments, actual measurement values per se and/or values of changes in those measurements may not necessarily be obtained or otherwise utilized. Instead, the fact that a change has taken place, at least a change having the sufficient magnitude relative to that which previously existed, is utilized as a basis for the verification. In an exemplary embodiment, it could be that the impedance between the electrode(s) has a value of X during a first period of time when the artificial device is being inserted further into the human. (And it is noted that the impedance could be a direct reading or could be and extrapolated reading based on voltage and/or current, utilizing Ohm's law, or power equations, etc. That is, impedance may not necessarily be read per se, but instead, readings that permit extrapolation or otherwise the termination of such can be obtained, which are then correlated to impedance values.) This first period of time can correspond to the time period where one or more of the electrodes of the conduit and/or termination, etc., are located in the middle ear. Then, upon puncturing the round window, and at least one of the electrodes being exposed to perilymph (or a coating thereof dissolving, reacting, for example, etc.), the impedance changes from X or otherwise is no longer X. This could be utilized as part of the verification or as the foundation of the verification. The values of X and the new value may not necessarily be known to the person (or machine, more on this below) executing that method. It could be enough that a change is taking place, which changes detected or otherwise determined to exist, to base the verification that the portion of the artificial device has entered the fluid containing cavity. Of course, specific readings could exist as well/be available to the person and/or the machine that is analyzing the results). In an exemplary embodiment, the impedance upon entering the fluid containing cavity could change to Y, where X is at least and/or equal to 0.1, 0.2, 0.3, 0.4, 0.5, 0.6, 0.7, 0.8, 0.9, 1. 1.25, 1.5, 1.75, 2, 2.25, 2.5, 2.75, 3, 3.5, 4, 4.5, 5, 5.5, 6, 6.5, 7, 7.5 8, 8.5 9, 9.5, 10, 15, 20, 25, 30, 35, 40, 50, 60, 70, 80, 90, 100, 150, 200, 250, 300, 400, 500, 750, 1000, 1500, 2000, 3000, 4000, 5000, 10000, 20 thousand, 30 thousand, 50 thousand or more, or any value or range of values therebetween in 0.01 increment times greater than or less than Y. A change within any of those ranges, such as a change that results in X being 0.1, 0.2, 0.3, 0.4, 0.5, 0.6, 0.7, 0.8, 0.9, 1. 1.25, 1.5, 1.75, 2, 2.25, 2.5, 2.75, 33, 4, 5, 6, 7, 8, 9, 10, 15, 20, 25, 30, 35, 40, 50, 60, 70, 80, 90, 100, 150, 200, 250, 300, 400, 500, 750, 1000, 1500, 2000, 3000, 4000, 5000, 10000, 20 thousand, 30 thousand, 40 or 45 thousand or more times, or any value or range of values therebetween in 1 increment greater than or less than Y can be indicative of the electrode(s) being in the presence of perilymph (or whatever substance is being used for the determination).

A 3 or 5 or 10 or 15 percent change in impedance can be significant enough to indicate contact with perilymph. And as seen above, there can be impedance decrease or increase. If the electrode is kept dry going through the middle ear and becomes in contact with perilymph the impedance will decrease significantly which is utilitarian, such as by way of example only, in combination with visual confirmation to be in the correct place (e.g., cochleostomy of the promontory of the cochlea). In an exemplary embodiment, the dry to wet impedance drop is used for distinguishing between contact with perilymph or contact with blood with an electrode shield or coating that only dissolves in the presence of perilymph or ion selective coatings on at least two electrodes that are brought in contact with perilymph to get the ratio of two impedance readings unique to perilymph as described above.

In an exemplary embodiment, at least two electrodes (one or both of which can be with the biosensor coating) are immersed in artificial perilymph such as biomarker free, physiological saline or a conductive hydrogel coating when approaching the cochlea, and then the contact with real/natural perilymph changes the impedance (and/or can be the trigger for the biomarker embodiments, etc.). Some embodiments include flooding the middle ear with physiological saline to wet the electrodes before entering the cochlea. The impedance between electrodes is relatively low and dominated by the physiological salinity of the environment. When brought into contact with blood, in some embodiments, little to nothing changes due to the same physiological salinity, but when in contact with perilymph, the perilymph specific biomarker molecule (e.g., protein) will bind to the biomarker specific coating causing a characteristic increase (not decrease) in impedance. Some embodiments use specific binding of the biomarker increases the impedance of a coating/at the coating.

Note also that in an exemplary embodiment (such as where the electrodes are kept dry before bringing in contact with perilymph, or otherwise do not come into contact with various substances that can change impedance), X is 100 kiloohms to 10 megaohms, and Y is 100 to 30 thousand ohms for a voltage of 0.25, 0.5, 0.75, 1, 1.25, 1.5, 1.75, 2, 2.5, 2.75, 3, 3.5, 4, 4.5, or 5 or 10 or 20 or 30 Volts or any value or range of is therebetween in 0.01 V increments) and a current of 0.01, 0.0.05, 0.1, 0.2, 0.3, 0.4, 0.5, 0.6, 0.7, 0.8, 0.9, 1.25, 1.5, 1.75, 2, 2.5, 3, 3.5, 4, 5, 6, 7, 8, 9, 10, 11, 12, 13, 14, 15, 16, 17, 18, 19 or 20 milliamps or any value or range of values therebetween in 0.001 mA increments. Note also that in an exemplary embodiment (where the electrodes are kept immersed in conductive media of physiological conductivity or salinity before bringing in contact with perilymph), X is 100 ohms to 30 thousand ohms, and Y is 0.1, 1, 2, 3, 4, 5, 6, 7, 8, 9, 10, 15, 20, 25, 30, 35, 40, 50, 60, 70, 80, 90, 100, 150, 200, 250, 300, 400, 500, 750, 1000, 1500, 2000, 3000, 4000, 5000, 10000, 20 thousand, 30 thousand, 40 or 45 thousand or more times, or any value or range of values therebetween in 1 increment greater than X, at a current of 0.01, 0.05, 0.1, 0.2, 0.3, 0.4, 0.5, 0.6, 0.7, 0.8, 0.9, 1.25, 1.5, 1.75, 2, 2.5, 3, 3.5, 4, 5, 6, 7, 8, 9, 10, 11, 12, 13, 14, 15, 16, 17, 18, 19 or 20 milliamps or any value or range of values therebetween in 0.001 mA increments. In an exemplary embodiment, the electrode surfaces exposed to the environment and/or the electrodes in their entirety are those of the same size as that of the Cochlear Nucleus 6 ™ and/or Nucleus 7 ™ and/or Nucleus 8 ™ cochlear implants supplied by Cochlear LTD as is present or otherwise available in the United States of America (as approved by its FDA for example) and/or the United Kingdom and/or the Republic of France and/or the Federal Republic of Germany, and/or the Commonwealth of Australia and/or New Zealand and/or the People's Republic of China on Jul. 4, 2021.

In at least some exemplary embodiments, signal characteristics of a signal from sensor components, such as the electrodes 844, are utilized to implement at least some of the teachings detailed herein, such as the teachings associated with determining and/or estimating with a high likelihood, etc., a location of a distal portion of a therapeutic substance delivery device. Embodiments utilize relatively low-energy that is measured. By way of example only and not by way of limitation, the energy levels utilized in some exemplary embodiments are energy levels below that which would otherwise result in a hearing percept in a 50 percentile male and/or female in accordance with the disclosure above if located immediately adjacent the modiolus wall of the cochlea (i.e., the energy levels are below a threshold level for such people). In at least some exemplary embodiments, a biphasic “stimulation” is utilized. Although in other embodiments, a tri-phasic or multiphasic regime can be utilized. The frequency can be between 10 and 1000 milliseconds or any value or range of values therebetween in 1 ms increments.

In some embodiments, impedance spectroscopy is conducted by measuring impedance by applying a sinusoidal electrical signal (AC) through the electrodes into the tissue at various frequencies and recording the electrical response (how the input signal is changed by going through the tissue). Data acquisition software and/or hardware is used to collate the data measured at various frequencies to plot an electrode impedance spectrum. Thus, embodiments measure electrical impedance using an AC signal at a one or many frequencies.

In some embodiments, “electrochemical impedance spectroscopy” (EIS) is used using the electrodes/device.

It is noted while at least some embodiments contemplated the utilization of alternating current, other embodiments can utilize direct-current, and thus the values that are measured or otherwise determined could be resistance as opposed to impedance. Herein, any disclosure of one corresponds to an alternate disclosure of the other unless otherwise noted providing that the art enable such.

Accordingly, the method can be executed based on the end result measurement raw values, the difference between the initial values and the end result values (absolute difference and/or percentage) and/or simply a value that is above a certain threshold. In an exemplary embodiment, the electrical phenomenon at issue in method action 1520 is a change in a value indicative of the electrical phenomenon.

Note further that in exemplary embodiments where the method is automated, a go/no go regime can be utilized where a device that is being utilized to monitor the impedance, or otherwise monitor the electrical phenomenon measurements or readings or data indicative of the electrical phenomenon at the working end of the artificial device can indicate such to a user of the artificial device utilizing automated algorithm that evaluates the readings in any of the aforementioned manners or other manners that can have utilitarian value to implement the teachings detailed herein. In an exemplary embodiment, the artificial device could be in signal communication with a computing device or the like or some form of annunciating device and the artificial device can provide an indication to the user indicating that the device is verified that a portion of the artificial devices entered the fluid containing cavity of interest. It is noted that the electronics package 849 and/or the computer 899 can be programmed or otherwise configured to execute any one or more of the method actions detailed herein, at least with respect to the evaluation and/or the indication and/or the control of an initiation of therapeutic substance delivery.

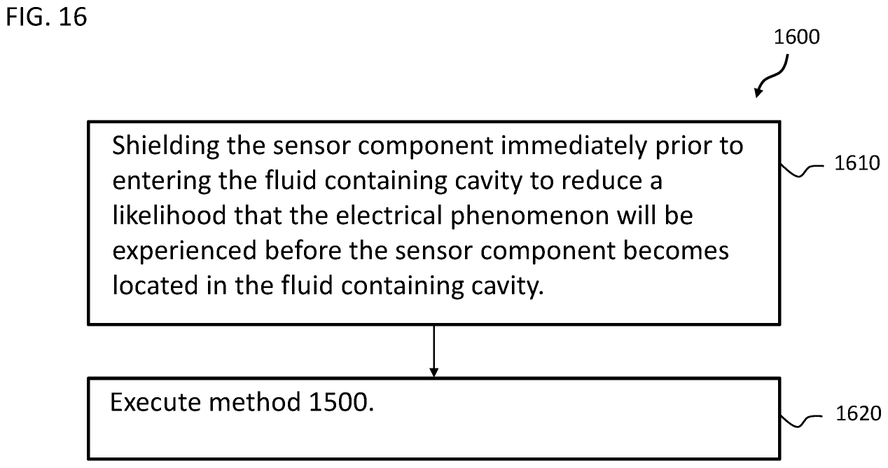

Concomitant with the teachings detailed above, FIG. 16 provides an exemplary flowchart for an exemplary method, method 1600, that includes method action 1610, and method action 1620, where method action 1620 entails executing method 1500. With respect to method action 1610, this includes the action of shielding the sensor component so that the sensor component is shielded immediately prior (which includes a scenario where the sensor component has been shielded for a long time—this simply requires that the sensor component is shielded during the aforementioned time and does not exclude shielding that occurs before) to entering the fluid containing cavity to reduce a statistical likelihood that the electrical phenomenon will be experienced before the sensor component becomes located in the fluid containing cavity. In an exemplary embodiment, there is a method of shielding the sensor component where there is the action of shielding the sensor component so that the sensor component is shielded for at least 1, 2, 3, 4, 5, 6, 7, 8, 9, 10, 20, 30, 40, 50, 60 seconds, or minutes, and/or hours prior to the sensor component becoming located in the fluid containing cavity. Again, this does not require that the action of shielding include an unshielded sensor component prior to the action of shielding. This requires that there be shielding present in the aforementioned time periods.

Accordingly, in an exemplary embodiment, method 1500 and/or 1600 includes a method where the sensor component is shielded immediately prior to entering the fluid containing cavity to reduce a statistical likelihood that the electrical phenomenon will be experienced before the sensor component becomes located in the fluid containing cavity.

Corollary to the above is that in an exemplary embodiment, there are method actions that are executed that result in or otherwise corresponds to shielding the sensor component. This is executed after the sensor component becomes located in the fluid containing cavity or prior to entering the fluid containing cavity. In an exemplary embodiment where the sensor component is shielded immediately prior to entering the fluid containing cavity, the on shielding takes place during a time period that is between the time period where the sensor component is shielded immediately prior to entering, and the entrance into the cavity. Thus, the time period associated with immediately prior to entering the fluid containing cavity can include both a subtime period where the component is shielded and a subsequent subtime period where the component is unshielded.

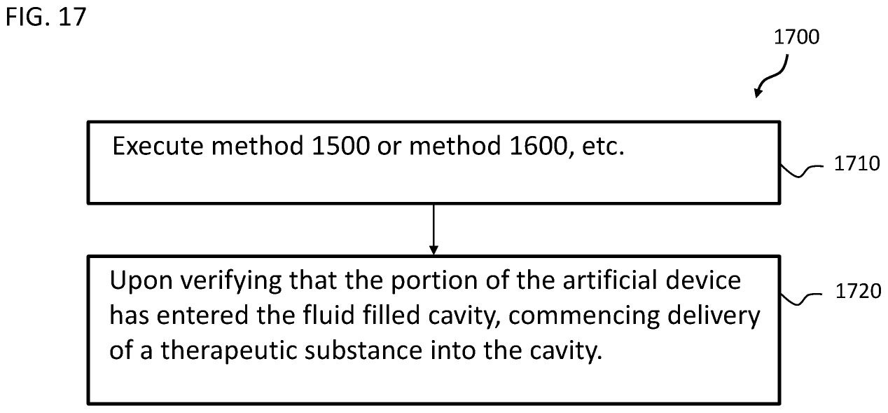

FIG. 17 presents an exemplary flowchart for an exemplary method, method 1700, that includes method action 1710, which includes executing method action 1500 and/or method action 1600, etc. Other variations of these methods can be executed in other embodiments as well. Method 1700 further includes method action 1720, which includes, upon verifying that the portion of the artificial device has entered the fluid filled cavity, commencing delivery of a therapeutic substance into the cavity. This can be done utilizing an automated delivery device. By way of example only and not by way of limitation, the electronics package 849 and/or the laptop 899 can be programmed and otherwise configured to analyze the data obtained from the sensor component(s) and upon verification that sensor component is in the fluid containing cavity, control the delivery device to initiate the delivery of therapeutic substance into the fluid filled cavity.

It is noted that with respect to automated devices, it is not necessary for the machine to actually know that the sensor component is located in the fluid containing cavity. In this regard, the action of verifying that a portion of the artificial devices entered the fluid containing cavity can be executed based on an analysis of data where the device is programmed or otherwise configured to read the data and execute additional actions if the data corresponds to certain values as opposed to other certain values for example.

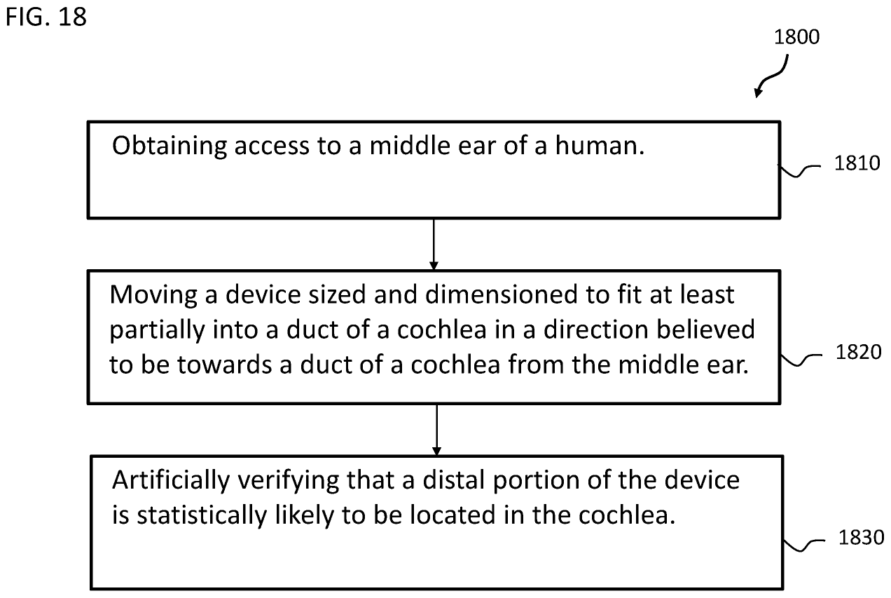

FIG. 18 presents an exemplary algorithm for an exemplary method, method 1800, which includes method action 1810, which includes obtaining access to a middle ear of a human. This can be executed by placing an incision in the tympanic membrane and extending a device through the incision and/or can be executed as a result of a passage that has been already placed in the tympanic membrane, such as through an orifice in a grommet in the tympanic membrane, which can be sealable or unsalable. Other routes into the middle ear can be executed, such as, for example, by making an incision in back of/behind the pinna area, and then drilling through the skull to reach the middle ear cavity. Any method of executing method action 810 and/or a device and/or system of doing so can be utilized in at least some exemplary embodiments.

Method 1800 further includes method action 1820, which includes the action of moving a device sized and dimensioned to fit at least partially into a duct of a cochlea in a direction believed to be towards a duct of a cochlea from the middle ear. This can be the conduit 313 and/or the termination 860 or any other device that can enable the teachings detailed herein. Method action 1800 further includes method action 1830, which includes the action of artificially verifying that a distal portion of the device is statistically likely to be located in the cochlea. In an exemplary embodiment, the action of verifying can be based on the sensor components coming into contact with the perilymph in accordance with the teachings detailed above and/or below. For example, when the impedance between the electrodes 844 of the embodiment of FIG. 13 reduces a significant amount and/or by a predetermined amount by more than a predetermined amount, etc., it can be deduced that it is statistically likely that the distal portion of the device at issue is located in the cochlea. This is because the perilymph within the cochlea will reduce the impedance between the electrodes when the perilymph comes into contact with the electrodes. In an exemplary embodiment, the action of verifying can be based on latent variables. This as compared to, for example, visually determining that the distal end of the device has entered the cochlea.

Note that artificially verifying that the device is statistically likely to be located in the cochlea is different than artificially verifying that the device is located in the cochlea. For example, utilizing an X-ray machine or a CT scan or some other imaging arrangement (which do not rely upon latent variables) would provide certainty or effective certainty of the location of the distal portion of the device. Conversely, relying upon the electrical phenomenon for example does not provide certainty or effective certainty, but can provide statistical likelihood. By way of example only and not by way of limitation, there could always be, for example, a 5% chance or a 1% chance that some form of body fluid other than perilymph has reduced the impedance between the electrodes. Thus, a reduction in impedance will not provide the effective certainty that would be akin to the aforementioned visual images for example, where the image is almost certainly a true reflection of the status of the distal portion. Granted, there could be errors of interpretation, but that is different than statistical likelihood. By rough analogy, a video of a car accident provides certainty that there was a car accident, whereas glass on a road accompanied by skidmarks and potentially a portion of a bumper one the road provides a statistical likelihood that a car accident took place.

Consistent with the teachings detailed above, in an exemplary embodiment, the device is a therapeutic substance delivery device, and the therapeutic substance delivery device carries sensor components, such as, for example, the electrodes 844. The sensor components are used in method 1800 to obtain data indicative of location and/or lack of location of the distal portion in the duct of the cochlea. In an exemplary embodiment, the obtained data is used to execute the action of artificially verifying. With respect to obtaining data indicative of location of the distal portion in the duct of the cochlea, this can correspond to a reading that indicates that the impedance has dropped a predetermined amount and/or large amount, etc. With respect to obtaining data indicative of a lack of location of the distal portion in the duct of the cochlea, this can correspond to a reading that indicates that the impedance remains high or otherwise has not changed a significant amount and/or a large amount.

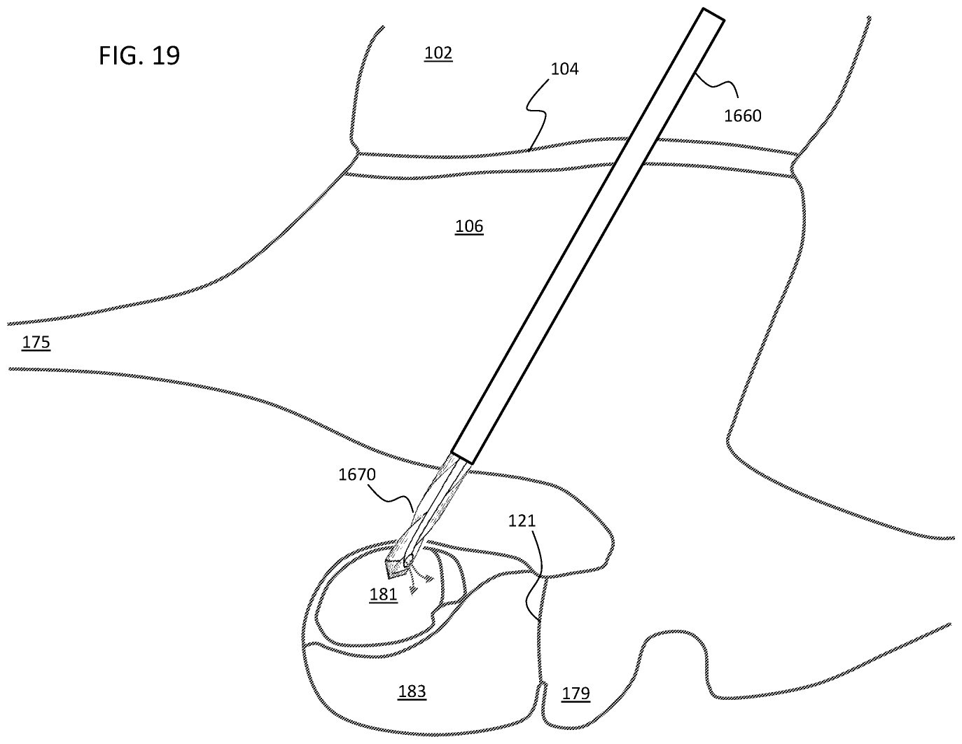

While the embodiments up to now have tended to focus on therapeutic substance delivery devices, other embodiments can include different devices, such as for example, a drill bit, although it is noted that the two are not mutually exclusive because here, we introduce the concept of a drill bit that is also utilized as part of a therapeutic substance delivery system, but we note that in some embodiments, this need not be the case. The drill bit is simply a standalone drill bit. But with regard to the combined embodiment, FIG. 19 depicts another exemplary embodiment for accessing the inner ducts of the cochlea. Here, the termination 1660 of the ear system therapeutic substance delivery device and/or endoscope (the drill bit could be part of an endoscope that does not have therapeutic substance delivery capabilities for example) includes a drill bit 1670. In an exemplary embodiment, the entire termination 1660 or at least the portions extending through the tympanic membrane to the distal end thereof is configured to be rotated at a speed that can enable the drill bit 1670 to be used to perform a cochleostomy as shown in FIG. 18. In some embodiments, it is only the drill bit that is rotated, and in other embodiments, it is the entire or most of the termination 1660. In an exemplary embodiment, the drill bit can be part of an assembly or structure that extends through the lumen of the termination 860. That is, in an exemplary embodiment, the termination 860 above can be utilized to pierce the tympanic membrane 104, and then the tip of the termination 860 can be moved to the promontory, and in an exemplary embodiment, the sharp tip can be utilized to stabilize the termination 860, and then the drill bit apparatus can be extended through the lumen of the termination 860, and then upon reaching the tissue of the promontory of the cochlea, the drill bit can be turned so as to drill into the promontory and thus reach a duct of the cochlea as shown in FIG. 19. In an exemplary embodiment, the scala vestibule is reached. That said, in an alternate embodiment, the termination 1660 is utilized to puncture the tympanic membrane and to drill through the promontory to reach the duct of the cochlea. In an exemplary embodiment, the tip of the drill bit is sufficiently sharp so as to pierce the tympanic membrane without turning the drill bit, while in other embodiments, the drill bit is turned to pierce the tympanic membrane. In another drill location on the promontory further away from the round window, it is possible to enter scala tympani using the drill. Embodiments can include using the teachings herein in/to reach any of the ducts of the cochlea and/or the semicircular canals, or other ducts of other organs.

Accordingly, in an exemplary embodiment, the ear system endoscope can have a drill motor associated there with to turn the termination 1660. Accordingly, in an exemplary embodiment, the surgical port 830 can receive a drill bit apparatus that extends from a drill motor of a surgical tool located outside the ear system endoscope, which drill bit apparatus extends through the conduit of the termination 860 and wherein the systems are configured so that the drill bit can extend outside of and pass the end of the termination 860 so that drilling can commence.

In view of the above, it can be seen that in an exemplary embodiment, there is the action of turning a drill bit having a component of the drill extending through the tympanic membrane. In an exemplary embodiment, the drill is rotated at more than or equal to 300, 400, 500, 600, 700, 800, 900, 1000, 1100, 1200, 1300, 1400, 1500, 1600, 1700, 1800, 1900, 2000, 2250, 2500, or 3000 RMP or more, or any value or range of values therebetween in 10 RPM increments and this can be done while the drill extends the tympanic membrane,

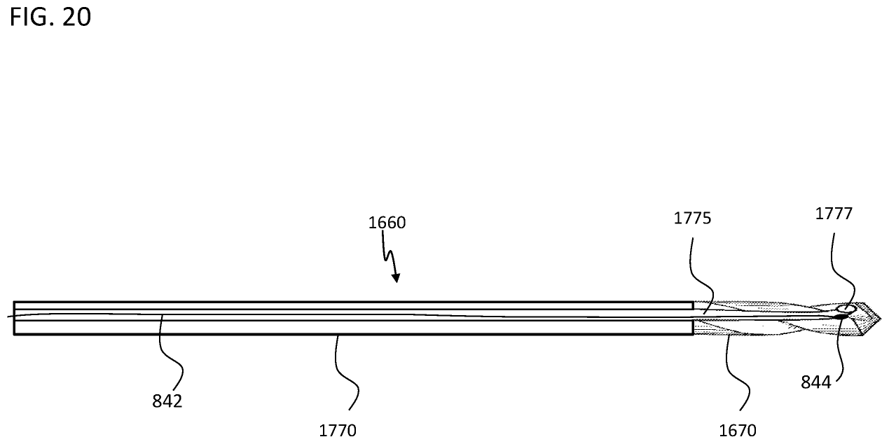

FIG. 20 depicts a cross-section of the termination 1660 of the embodiment of FIG. 19. As seen, there is a conduit 1775 extending through the termination, terminating at an orifice 1777. In an exemplary embodiment, the conduit and the orifice are utilized to provide irrigation and/or cooling fluid during the action of drilling to the ducts of the cochlea. In addition, or alternatively, the conduit and the orifice can be utilized to deliver a therapeutic substance into the duct(s) of the cochlea upon drilling through to the duct(s) of the cochlea. This is represented by the curved arrows emanating from the orifice shown in FIG. 19.

In an exemplary embodiment, the drill bit includes at least one electrode 844 as shown in FIG. 20. The electrode 844 is connected to a lead 842 in a manner concomitant on with the embodiments detailed above, although in other embodiments, this can be different. The electrode 844 is located proximate the orifice 1777, just inside the orifice as can be seen. In some embodiments, the electrode can be located to the orifice or even at the opening of the orifice, while other embodiments the electrode can be located further into the passageway of the conduit 1775. While this embodiment does not include a second separate electrode per se, other embodiments can include another electrode, such as electrode space in the opposite side of the conduit 1775 within the passageway and also connected to an electrical lead. In this embodiment, the electrically conductive properties of the tip of the drill bit are utilized as the “second electrode.” The conductive body of the termination can be utilized as the return, while in other embodiments, a separate lead can be snaked through the passageway from the location proximate the orifice 1777, where the electrical lead could be electrically connected to the material of the drill bit at that location so that the electrical lead is utilized as the return as opposed to a larger portion of the body that establishes the termination. In an exemplary embodiment, the electrical lead could be placed into electrical conductivity with the remainder of the drill bit at a location just inboard of the most distal portion of the drill bit tip, so that at least a portion of the sensor system is located as distally as possible (where any electrical resistance associated with the drill bit material is reduced in theory as much as possible because the electrical lead is as forward/distal as possible).

While this embodiment depicts a conduit 1775 concomitant with the nature of the drill bit as being utilized as part of a therapeutic substance delivery device, in other embodiments, the drill bit may not have conduit 1775. In an exemplary embodiment, one or more electrodes could be located on the outer surface of the drill bit at the distal end thereof and/or proximate the distal end thereof. Any device, system, and/or method that can enable the implementations of the teachings detailed herein with the drill bit can be utilized in at least some exemplary embodiments.

The working principle with respect to the embodiment of FIG. 20 is that when the drill bit drills through the bony structure and enters into the duct 181, the perilymph therein will come into contact with the electrode(s) and thus reduce the impedance in the manner concomitant with the embodiments detailed above. When this embodiment is utilized with method 1800, this can be utilized to determine that the distal portion of the device, here, the drill bit, is statistically likely to be located in the cochlea.

Thus, in an exemplary embodiment, the device of method 1800 can be a drill bit, where the drill bit carries sensor components, such as, the electrode(s) (and where the use of the body of the drill bit is a sensor component) and the sensor components are used to obtain data indicative of a location and/or lack of location in the duct of the cochlea, where the obtained data is utilized in at least as part of the action of artificially verifying.

In an exemplary embodiment, the device used in method 1800 carries and/or includes a substance that chemically reacts with another substance in the duct of the cochlea, and method 1800 includes having the substance chemically react with the another substance in the cochlea and obtaining data indicative of the chemical reaction having taken place, and the action of obtaining data indicative of the chemical reaction is used at least as part of the action of executing the action of artificially verifying. In an exemplary embodiment, the substance that chemically reacts can correspond to the coating 1444 detailed above. This chemical reaction could result in an increase or a decrease in the conductivity of the coating 1444, and thus would permit a decrease or an increase in impedance between the electrodes via the perilymph for example. In an exemplary embodiment, there is selective coating(s) of one or more electrodes to result in a decrease or increase in conductivity resulting in a characteristic increase or decrease in impedance between the two electrodes. And in this regard, it could be that only one of two electrodes is coated for example (or otherwise has the impedance varying features as detailed herein).

The substances in the duct of the cochlea to which the substance chemically reacts can be substances that are found in effective quantities to implement the teachings herein in the above noted human factors engineering populations by way of example only and not by way of limitation, which substances can be natural, but also the teachings detailed herein can be substances that are artificial, or otherwise not normally found in ducts of the cochlea, such as, for example substances corresponding to therapeutic substances or steroids, etc., which substances could be a result of medications being taken by the person/human possessing the cochlea, etc.).

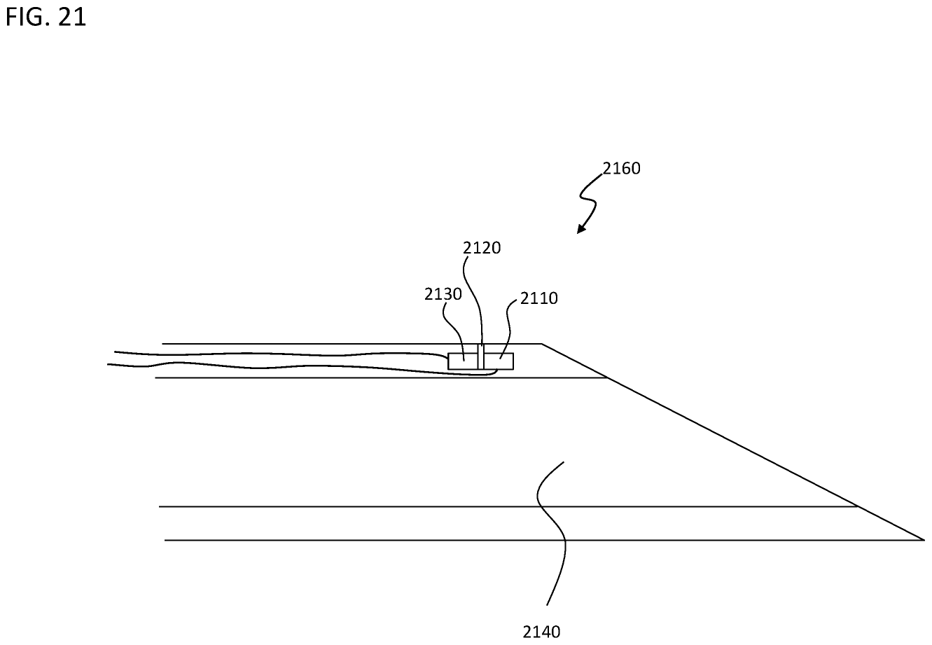

For the most part, the embodiments detailed above rely on the utilization of the body fluids to provide the reduction in impedance between the electrodes. But other embodiments can use different principles, at least with respect to the conductivity. FIG. 21 presents an exemplary distal end of a termination 2160, that can be utilized to pierce the round window of the cochlea. Here, there is a lumen 2140 concomitant with a termination, along with sensor components located in the wall of the termination/the wall establishing the lumen 2140. Here, we see two electrodes 2110 and 2130 that are electrically isolated from each other owing to insulation (not shown) and the properties of the barrier 2120, which is in physical contact with the electrodes. Perilymph or other body fluids cannot reach the electrodes 2130 and 2110. What does happen in this exemplary embodiment is that the barrier 2120 is made of a substance that chemically reacts with perilymph/substances within perilymph/making up perilymph. In an exemplary embodiment, barrier 2120 does not chemically react with water for example or body fluids that might be found in the middle ear region. In an exemplary embodiment, upon barrier 2120 coming into contact with the perilymph in the cochlea at the outermost surface thereof, the surface exposed to the outside of the termination 2160, a chemical reaction takes place in the material the barrier 2120 that transforms the material into a more conductive state, thus placing the electrodes 2110 and 2130 into electrical conductivity or otherwise reducing the impedance between those two electrodes. This can thus be utilized obtain data indicative of the chemical reaction, and thus can be utilized as least as part of the action of executing the action of artificially verifying of method 1800.

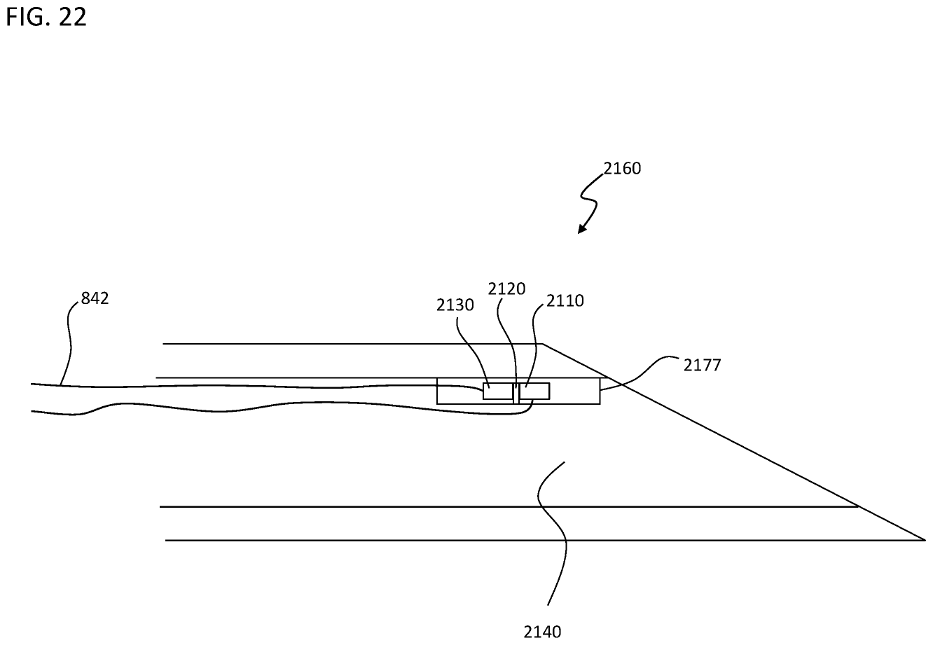

While the embodiment depicted in FIG. 21 presents the sensor components located in the wall of the termination 2160, in other embodiments, the sensor components can be located on the outside and/or on the inside of the termination. Moreover, in an exemplary embodiment, the sensor components can be located as part of a module or the like so that the sensor components can be more easily attached to the termination/structure establishing the lumen 2140. FIG. 22 presents an exemplary embodiment of a module 2177 that includes a housing that supports and otherwise contains the electrodes and the barrier 2120, where the leads can be snaked through the lumen 2140. In an exemplary embodiment, the module 2177 can be inserted into the lumen through the lumen and attached at a location that has utilitarian value, such as that is shown. This can have utilitarian value with respect to not having to modify or otherwise adjust the structure establishing the lumen 2140. In an exemplary embodiment, an interior sleeve having a hollow portion for the electrodes can be used. The sleeve, as with the housing of the module 2177, can electrically isolate the electrodes as utilitarian. And while the embodiment depicted in FIG. 22 utilizes the chemically reactive barrier, other embodiments can utilize this concept with respect to the electrodes that are simply exposed to the perilymph for example. That is, in module 2177 can have a platform that supports the electrodes and enables simple positioning of the electrodes.

It is to be understood that in some exemplary embodiments, negative pressure can be utilized to draw perilymph into the lumen, such as in embodiments where the electrodes are located inside the lumen 2140. By way of example only and not by way of limitation, if the termination is part of a syringe, the plunger of the syringe can be pulled backwards so as to drawl perilymph into the lumen, if present. If no perilymph is present, the impedance would remain the same or otherwise would not decrease to values indicative of distal portion of the device utilized in method 1800 being located in the duct of the cochlea or whatever cavity is the subject of the method.

It is to be noted that in at least some exemplary embodiments, the location of the electrodes can be varied relative to the distal end of the device being utilized. This can have utilitarian value with respect to ensuring that the entire distal end is located in a duct of the cochlea for example. In this regard, say the electrodes of FIG. 22 were instead further located to the right, and located on the bottom of the lumen instead of the top of the lumen as shown, it could be that the impedance drop would exist without the entire opening of the lumen 2140 being located in the duct of the cochlea. Accordingly, by replacing the electrodes at precise locations along the termination, the methods herein can be implemented and the devices can enable a determination that the entire working end of the device is located in the cochlea, as opposed to a scenario where only a portion thereof is located in the cochlea. Thus, embodiments include methods where the action of artificially verifying includes the action of artificially verifying that all of the opening/delivery opening of a termination or otherwise a therapeutic substance delivery device is statistically likely to be located in a duct of a cochlea or otherwise in a cavity of interest. This as opposed to a verification where the verification may or may not correspond to the entire opening being located in the cochlea duct.

Another embodiment includes first electrode pair with one or both electrodes of that pair being at the tip or at a more distal location of the termination or whatever device being inserted into the cavity which is used to confirm contact with perilymph. This is conveyed to the handler or person operating the method/using the device. The termination (or other device) can be continued to be advanced until a second electrode of a second pair of electrodes (or both) is contacting perilymph to indicate the termination (or other device) is advanced deep enough into the duct (based on a predetermined concept) and advancement can then be stopped. This would thus include two feedback signals, one to confirm good location and a second one to stop advancing the tool. Moreover, three, four, five, six or more electrode pairs (or single electrodes) staggered in a utilitarian manner along a length of the termination (or other device) can be used to determine a depth (as opposed to a go/no-go concept). As the staggered electrodes come into contact with the perilymph, signals are generated/changed, etc., which signals can be used as an indicator of depth, etc.

Thus, in some embodiments, at least one or two or all of the electrodes or otherwise sensor component(s) and/or sensor component(s) that are exposed to the environment are located within 1, 2, 3, 4, 5, 6, 7, 8, 9, 10, 11, 12, 13, 14, or 15 mm, or any value or range of values between any of those values in 0.1 mm increments from the most distal portion and/or from the most distal opening of a therapeutic substance delivery orifice and/or from the most proximal opening of a therapeutic substance delivery orifice of the device utilized to execute method 1800.

Consistent with the concept of utilizing a substance that chemically reacts with another substance that is found in the duct of the cochlea, the substance, which could be a coating over an electrode, or some other coating, could be used as a biosensor to monitor impedance changes over time, such as, for example, during postoperative time periods. Further, some embodiments can utilize the coatings as biosensors to trace a therapeutic substance, and/or disease state. By way of example only and not by way of limitation, the electrode(s) could have a drug specific MIP coating to trace or track a drug or otherwise some form of therapeutic substance in order to determine or gage or otherwise estimate the amount of the therapeutic substance or drug at one or many locations in the cochlea (e.g., as a percentage of a volume of perilymph, for example). Further by way of example only and not by way of limitation, the electrodes could have a disease state specific feature. For example, the electrode(s) could have a histamine specific MIP coating to trace or track histamine to determine or gage or otherwise estimate the amount of histamine in one or many locations in the cochlea (e.g., as a percentage of a volume of perilymph, for example).

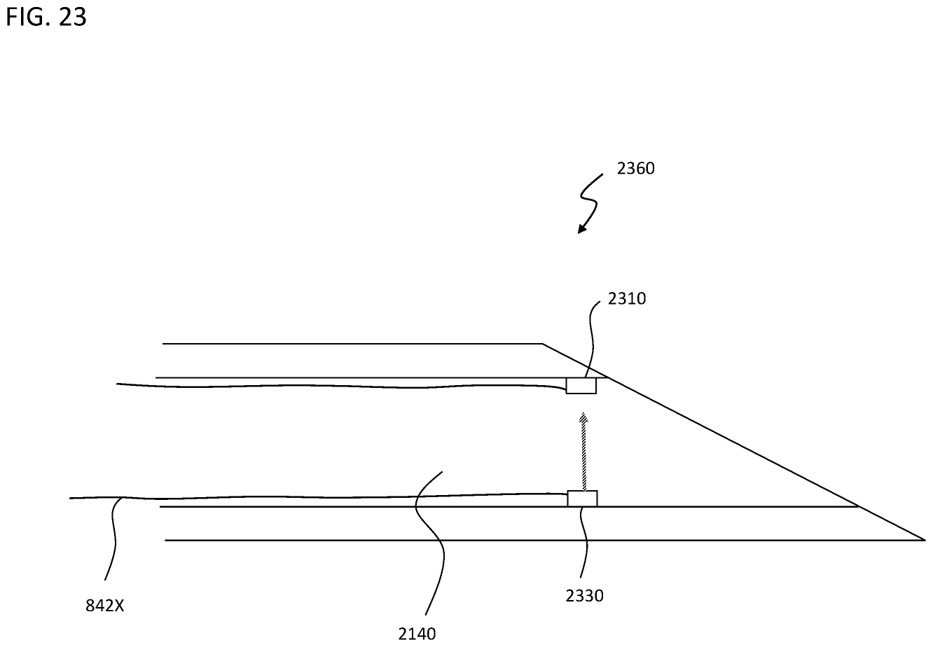

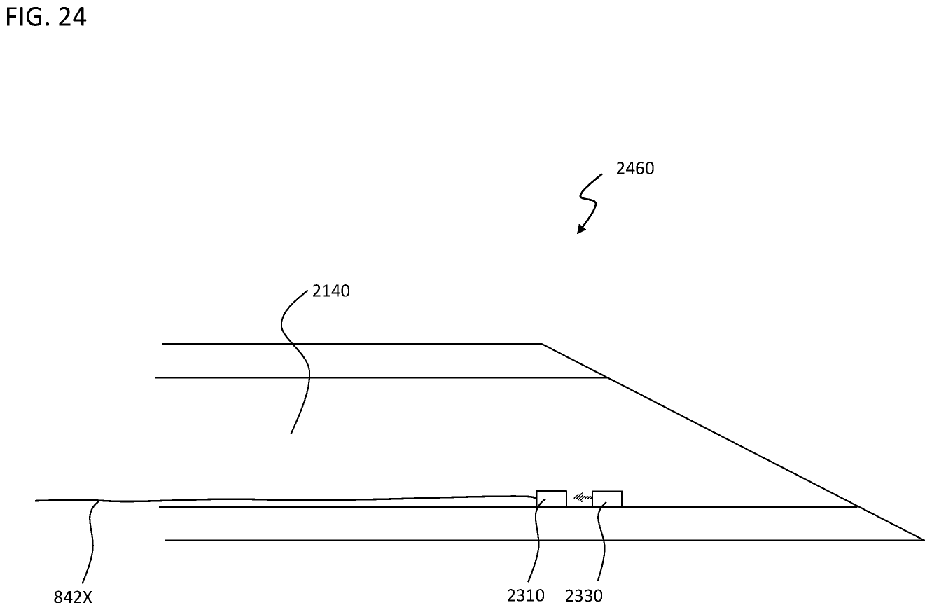

While the embodiments above have focused on materials and substances and coatings associated with electrodes, it is noted that in other embodiments, other types of sensor components are sensor arrangements can be utilized. FIG. 23 presents another exemplary embodiment that utilizes radiation sensors. Here, there is a termination 2360 an emitter 2330 that emits some form of radiation, which can be radiation in the visible spectrum, the emitted radiation represented by the arrow, and thus these devices can be like sensors, and there is a receiver 2310 that can receive light emitted from the emitter 2330. In an exemplary embodiment, the emitter and/or receiver, or more specifically, the working end of the emitter and/or the working end of the receiver (e.g., lens or transparent barrier) can be coated with some form of substance that chemically reacts with the substance in the duct of the cochlea that changes its property. In an exemplary embodiment, this changing property can be to make the substance more transparent or less transparent, and thus an increase or decrease in the magnitude of radiation received at the receiver 2310 can be utilized as an indication that the sensor components, and thus the distal end of the device, is located in the duct of the cochlea. While the embodiment depicted in FIG. 23 depicts an arrangement where the radiation beam traverses the diameter of the lumen 2140, in other embodiments, the receiver could be located elsewhere, such as shown in FIG. 24 with respect to termination 2460. Here, the radiation path has a much shorter distance to travel. The distance of the radiation path travels is irrelevant at least in some embodiments, owing to the fact that the controlling feature is a substance that changes properties in the presence of perilymph. And it is noted that here, standard electrical leads can be utilized, but also, element 842X could instead be fiber-optic cables, etc. And in some embodiments, the sensor components at the distal end of the device can be completely devoid of electrical components/conductive material.

And while the above embodiments have focused on raw changes to the magnitude of received radiation, other embodiments can utilize spectral shifts and/or other changes that occur as a result of the chemical reaction of the substance utilized with the sensor components. For example, the substance can have a chemical reaction that can change the resulting wavelength of the radiation that is received by the receiver relative to that which would otherwise be the case in the absence of the chemical reaction. Further, in varying degrees of chemical reaction and/or different chemical reactions can result in different changes in the properties of the radiation, thus providing more specific details of the fluid to which the sensor components have been exposed. For example, a first drug contained in the perilymph could result in a certain chemical reaction that results in a wavelength shift of one amount, and a second drug contained in the perilymph could result in a chemical reaction that results in a wavelength shift of another amount. The absence of drug could result in no chemical reaction and thus no wavelength shift.

Note further that in some embodiments, a chemical reaction based arrangement may not necessarily be relied upon or otherwise present. For example, the aforementioned light sensors can be configured so that the given drug results in a wavelength shift and/or an intensity change without any chemical reaction. Corollary to this is that in at least some exemplary embodiments, instead of a chemical reaction, the substance that coats the receiver for example could be a substance that simply dissolves, thus increasing the amount of radiation that is received by the receiver. The point is that the various techniques herein utilized as the working elements are not mutually exclusive with respect to the underlying phenomenon that can be utilized to implement the teachings detailed herein.

In an exemplary embodiment, the device used in method 1800 carries and/or includes a structure that accepts and/or accretes a substance located in the duct of the cochlea. Method 1800 further includes having the substance accepted by the structure and/or having the substance accreted by the structure and obtaining data indicative of the acceptance and/or accretion having taken place. Here, the action of obtaining data is used at least as part of the action of artificially verifying.

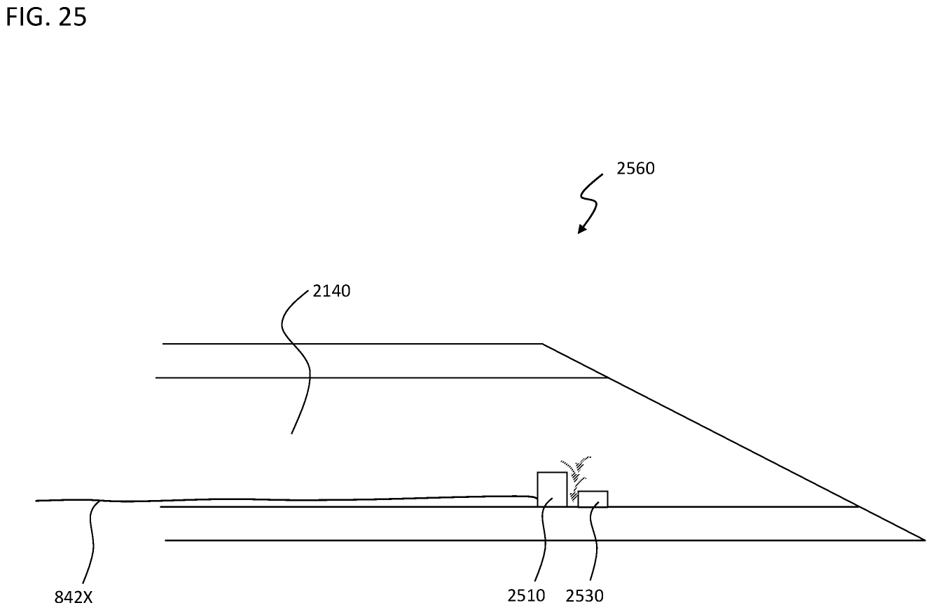

FIG. 25 presents sensor components 2510 and 2530 located inside a lumen 2140 of a termination 2560 according to an exemplary embodiment, which sensor components can correspond to the sensor components 2310 and 2330 detailed above, but can also be different types of sensor components as will be described in a moment. In this embodiment, the sensor components are configured to trap or otherwise accrete substances that are located in a cochlea/duct of the cochlea, or any other cavity or enclosure they can be the subject of the teachings detailed herein, between the two sensor components as indicated by the arrows. In an exemplary embodiment, the substances “fill” or otherwise become retained in the void between the two sensor components. In this embodiment, sensor component 2510 is taller than the sensor component 2530 so as to induce a potential eddy current or turbulence that will cause the substance to be more likely to accrete between the two sensor components. In an exemplary embodiment, this could change the intensity of the light radiation received by the receiver. In an exemplary embodiment where the sensor components are electrodes, the accretion of substance could increase the impedance between the two sensor components. All of this could be utilized in the action of artificially verifying that the distal portion of the device is statistically likely to be located in a cochlea for example.

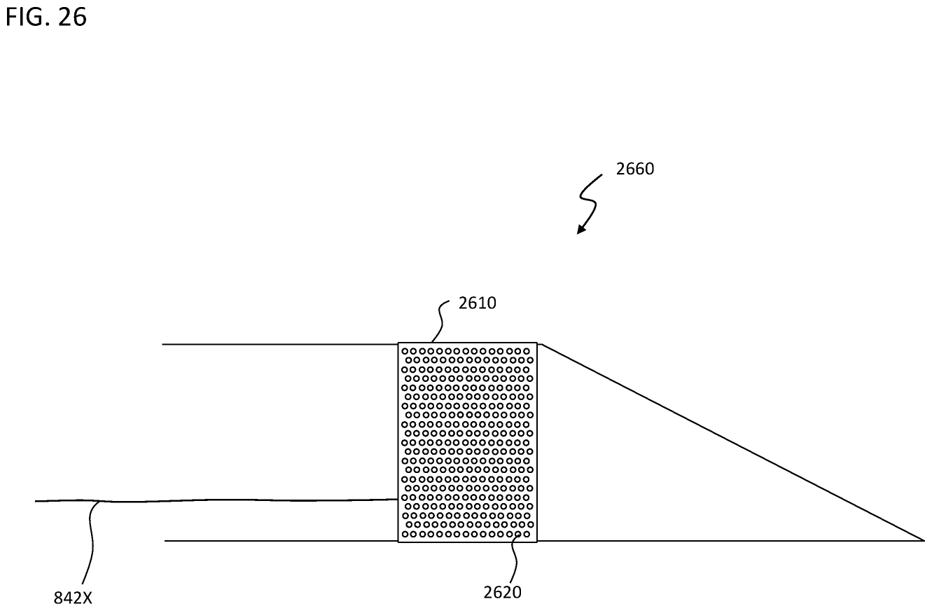

FIG. 26 depicts another exemplary termination 2660 according to an exemplary embodiment. Here, only the outside of the termination is shown. In this exemplary embodiment, there is a band 2610 that includes a series of pores 2620. That is, the outer surface of the band 2610 is porous. The band can be shrink fit or interference fit around the outer diameter of the body of the termination, or could be attached in any other utilitarian manner. And while the band is depicted on the outside, it is to be understood that this band could instead be deployed on the inside, such as along the inner walls of the lumen. Here, as the substance that is located in the cochlea accretes into the pores 2620 or otherwise is accepted and the pores, and electrical property of the band 2610 could change, which change or the value of the new reading could be utilized in the action of artificially verifying the method 1800. In an exemplary embodiment, it is proteins that accrete the pores, or otherwise become located in the pores, where the pores are sized and dimensioned or otherwise configured so that only certain proteins will accretes or otherwise become located in the pores, at least in a manner that can have utilitarian value with respect to the teachings detailed herein, which proteins are proteins that are typically found in the cochlea with respect to the 50 percentile male and/or female detailed above. In other embodiments, the pores are sized and dimensioned to accrete or otherwise permit the entrance of molecules related to substances that have been artificially provided to the human and thus find their way to the cochlea, such as steroids or anti-inflammation drugs, etc. And note while the above focuses on pores, in other embodiments, other types of cavities and/or other types of structure can be utilized providing such is enabled the accretion and/or capture or otherwise the location of the pertinent substances into such structure so that the teachings detailed herein can be implemented.

In an exemplary embodiment, the various proteins or otherwise the substances located in the cochlea “clog” the pores or otherwise to interface with the structure so as to increase an impedance in at least some exemplary embodiments. That is, the sensor component has a first impedance without the proteins or substances clogging the pores, and then as those proteins or substances, which are specific, clog the pores, which pores could not be clogged by other proteins or substances, or at least dissimilar proteins or substances, the impedance changes, and in this embodiment increases. The absolute value and/or the change of the impedance or any other phenomenon or feature the impedance can be utilized to determine the location of the therapeutic substance delivery device and to measure the concentration of the substance clogging the pores (more pores get clogged with higher concentration). In this regard, this is the principle of the MIP coatings mentioned above. The MIP coating has pores made by polymerizing the coating in the presence of the analyte. The analyte is then removed leaving behind a porous structure which can accept the analyte in question. The pores are filled depending on the concentration of analyte (affinity of the analyte to diffuse through the coating and into the pores) causing the more conductive fluid to be displaced out of the pores which causes an increase in impedance, by way of example.

In an exemplary embodiment, liners can be replaceable that can be utilized to capture or otherwise accrete a given substance, or otherwise can react with a given substance, to implement the teachings detailed herein.

In an exemplary embodiment, the readings can provide details regarding the concentrations of a given substance. This can be used for the determination of the location of the therapeutic substance delivery device. By way of example only and not by way of limitation, if a substance is commonly found in portions of the body, but that substance is found in a particular portion of the body that is of interest, in different concentrations, by utilizing a sensor that can determine with reasonable specificity the concentration, the location of the device can be determined at least relative to other possible locations.

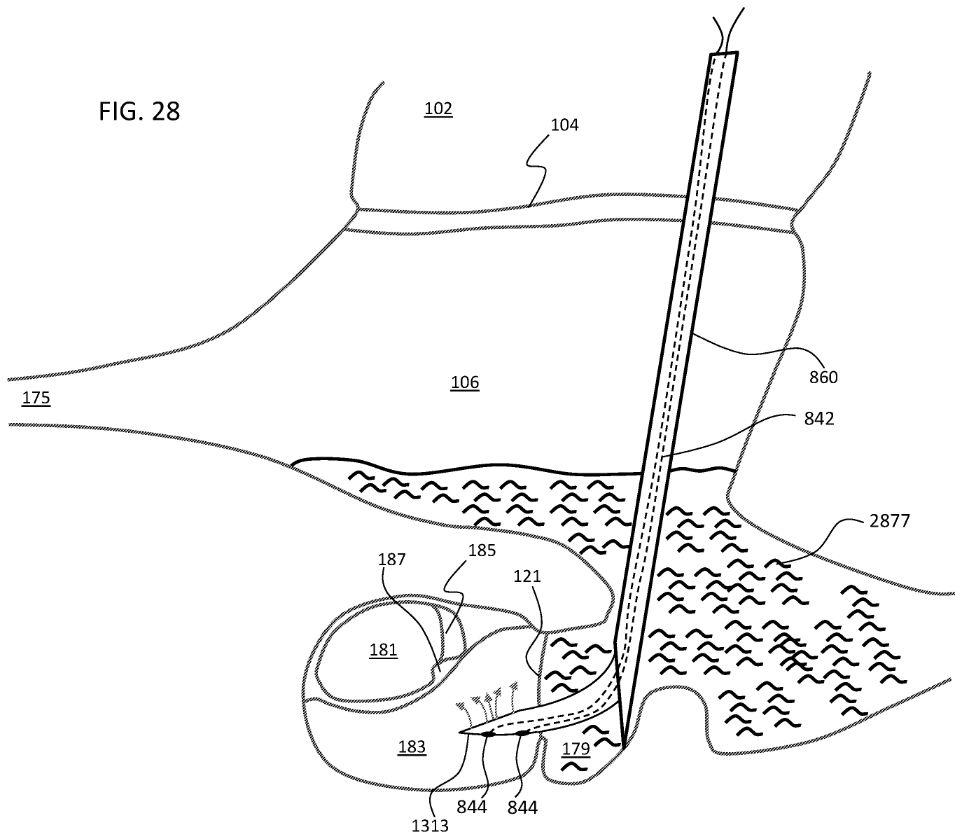

An exemplary embodiment utilizes a substance having a known conductivity and/or impedance and/or a substance that has a high impedance in areas outside the cavity at issue, such as outside the duct of the cochlea at issue, so as to more utilitarianly reduce the likelihood of false positive readings relative to that which would otherwise be the case. In this regard, FIG. 28 presents an exemplary scenario where a high impedance fluid 2877 partially fills the middle ear cavity 106. In an exemplary embodiment, this can be DI water. More particularly, an impedance drop between the electrodes can be unspecific and could also happen due to contact with other body fluids and/or other body tissues by way of example only. This scenario can be mitigated, or otherwise the likelihood of such can be reduced by keeping or otherwise establishing an environment in which the distal portion of the therapeutic substance delivery device is located immediately before making contact with perilymph for example, in a high impedance environment. Here, a low conductive medium such as DI water is utilized. Physiological saline can also be suitable as detailed above. A substance that can be mixed with perilymph without deleterious issues or otherwise issues that cannot be managed can be utilized in some embodiments. However, in an alternate embodiment, the area of the middle ear can be purposely dried or otherwise artificially dried beyond that which would otherwise be the case at least prior to insertion of the distal portion of the therapeutic substance delivery device into the middle ear and/or moving the distal portion proximate the ultimate target. With regard to the latter scenario, a person implementing at least some of the teachings detailed herein could deduce based on the amount of insertion of the therapeutic substance delivery device that the distal portion has not yet reached a location proximate the target cavity. For example, because the spatial geometry of a middle ear cavity would generally be known based on human factors engineering knowledge and/or an immediate visual inspection or a noninvasive inspection (e.g., X ray or CT scan), and because the length of the termination for example would be known, the person and/or machine implementing the teachings detailed herein would know that if the distance from the tympanic membrane to the round window of the cochlea is X, and only 0.7X of the termination has been extended through the tympanic membrane, any changes in impedance for example would be discounted (at least for purposes related to determining whether or not the distal portion of the termination has reached/entered the duct of the cochlea for example —a change in impedance could be utilized to indicate that the distal portion has come into contact with something that may not be desired to come into contact with such, and thus could be utilized as an indication to the person and/or machine executing the method to make an adjustment or otherwise stop the insertion procedure and readjust by way of example).

Accordingly, as seen in FIG. 28, we see that the middle ear cavity is around half filled (or may be a bit less by volume) with DI water 2877 the point is that the fluid 2877 does not fill the middle ear cavity, while in some embodiments, the fluid could fill the middle ear cavity. Thus, the electrodes 844 would be immersed in the fluid 2877 until piercing the round window 121, and thus the readings from the electrodes would indicate a low impedance relative to that which is experienced when the electrodes and/or the duct 183 can come into contact with the perilymph therein or otherwise indicate an impedance of a certain value that is different than the value that is expected when the electrodes come into contact with the perilymph.

And in an exemplary embodiment, the fluid 2877 can be “injected” via the termination 860 after the termination has pierced the tympanic membrane. In an exemplary embodiment, the termination 860 can have indicators indicating a distance from the tip of the termination. A user can compare the indicators to the location of the tympanic membrane and deduce approximately how far the distal tip has been inserted beyond the tympanic membrane. Note that these indicators could be electronic in nature or could be color-coded. Embodiments include indicators that can be read by a machine for an automated process to determine the length of the termination inserted through the tympanic membrane (and can use that in the above-noted scenario where the distance of insertion is deduced to discount for false positives). After a desired amount of the termination has been inserted, the fluid 2877 can be injected through the termination (including through the conduit 1313, or around the conduit), and when a sufficient amount of fluid has been delivered to the middle ear cavity, the insertion of the termination towards the round window can continue.

In an exemplary embodiment, irrigation regimes can be utilized to provide the fluid 2877 into the middle ear. By way of example only and not by way of limitation, irrigation port 840 can be utilized in at least some exemplary embodiments. For example, the irrigation port 840 can be placed into fluid medication with a supply of fluid that can have utilitarian value with respect to implementing the teachings detailed herein, and the fluid can be supplied in a manner concomitant with an irrigation process used in a surgery associated with accessing the cochlea.

Thus, there is a method, such as method 1800, that further includes the action of providing a fluid and/or a semi-fluid substance into the middle ear cavity, the substance having a known property (e.g., impedance/conductivity, such as DI water). The method further includes sensing a feature of the known property during a first time period (e.g., such as the time period that is less than and/or equal to 1, 2, 3, 4, 5, 6, 7, 8, 9, 10, 11, 12, 13, 14, 15, 20, 25, 30, 35, 40, 45, 50, 55, or 60 seconds, or any value or range of values therebetween in one second increments-note that this does not rule out sensing the feature during time periods before these time periods). The method also includes ceasing to sense the feature of the known property during a second time period following the first time period (the second time period can begin immediately after the above-noted ranges/periods of the first time period—the second time period can be immediately adjacent the first time period-indeed, the beginning of the second time period can call to an end the sensing during the first time period/can be the demarcation between the sensing of the first time period and the second time period). In an exemplary embodiment, the action of ceasing to sense the feature is used at least as part of the action of executing the action of artificially verifying. In an exemplary embodiment, the action of ceasing to sense the feature is totally a result of the sensor component leaving contact with her otherwise interfacing with the substance that caused the feature of the known property, such as the fluid and/or semifluid substance. In an exemplary embodiment, the substance can be a gel having a known conductivity and/or impedance. In an exemplary embodiment, the substance can be or include a therapeutic substance. In this regard, the two birds with one stone scenario can apply: the gel could provide a therapeutic substance that diffuses or otherwise leaches through the barriers between the middle ear and the inner ear while the gel is located in the middle ear and/or can provide therapeutic substance to the middle ear. In an exemplary embodiment, the gel is a high viscosity substance. In an exemplary embodiment, the gel has a viscosity of at least 10, 15, 20, 30, 40, 50, 75, or 100 times or more of the DI water and/or tap water.

In an exemplary embodiment, the end of the termination/distal end of the therapeutic substance delivery device could be covered with the gel. That is, by utilizing the viscosity of the gel, a sufficient amount of the gel can be carried by the therapeutic substance delivery device through the tympanic membrane an amount sufficient to enable the teachings detailed herein to have a utilitarian value. In some embodiments, the gel could be forced through the termination so that the gel surrounds the conduit released the gel lies over the electrodes that are located on the conduit, and the viscosity is utilized to “hold” the gel in place until the conduit enters the cochlea. In an exemplary embodiment, a nonconductive hydrogel can be utilized.

In an embodiment, ionic imbalances and/or sodium pump related features can be measured or otherwise used as a basis for at least some of the determinations herein, and thus embodiments include sensors that can sense such features and/or imbalances, etc., at least sufficiently to establish with the certainty detailed herein the location of the pertinent portion of the device.

Embodiments include switching on and/or off the sensor arrangement as needed. Thus, in an exemplary embodiment, the sensor arrangement may be active only for 10 or 20 or 30 or 40 or 50 or 60 or 90 or 120 or 240 seconds or any value or range of values therebetween in one second increments within an hour long period on either side of the activation. In an exemplary embodiment, the sensor arrangement is a predefined arrangement with fixed features. That is, for example, the voltage and/or frequency of operation does not change and/or is not adjustable.

An exemplary embedment includes a non-transitory computer-readable media having recorded thereon, a computer program for executing at least a portion of any one or more methods detailed herein and/or variations thereof. In an exemplary embodiment, the computer program includes code for analyzing input from a sensor indicative of electrical phenomena within a cochlea. The program also includes code for at least one of: (i) automatically indicating to a healthcare professional data based on data indicative of a sensor component being located in a cochlea based on an analysis executed using the code for analyzing; or (ii) automatically executing a therapeutic substance delivery action based on an analysis executed using the code for analyzing.

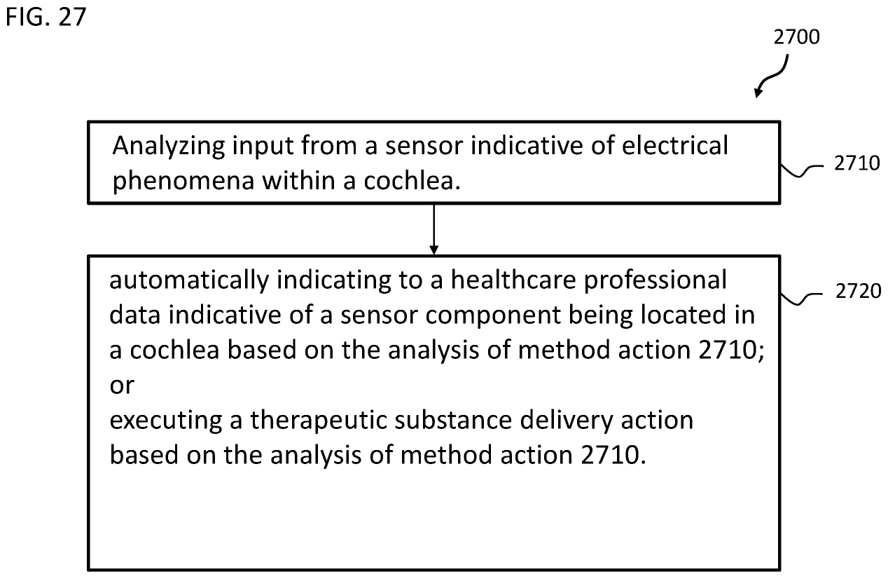

Briefly, it is noted that any disclosure herein of code for executing a method action corresponds also to a disclosure of a method having those method actions. Accordingly, in an exemplary embodiment, there is method 2700 as seen in FIG. 27, which includes method action 2710, which includes the action of analyzing input from a sensor indicative of electrical phenomena within a cochlea. Method 2700 also includes method action 2720, which includes the action of at least one of automatically indicating to a healthcare professional data based on data indicative of a sensor component being located in a cochlea based on the analysis of method action 2710 or executing a therapeutic substance delivery action based on the analysis of method action 2710. (Method action 2720 could be one or the other or could be both, and can also include other actions as well, and thus the code can be for one or the other or for both.) With regard to method action 2720, the action of automatically indicating to a healthcare professional data based on data indicative of a sensor component being located in the cochlea need not state such per se. The data is data based on data indicative of a sensor component being located in the cochlea if the data is based on the data. For example, what is provided to the healthcare professional can be a green or red light, or an audio signal. Or simply raw impedance data (which is data that is data based on the impedance signal—that includes the data that is based on the impedance signal). With regard to the action of automatically executing a therapeutic substance delivery action based on the analysis executed utilizing the code for analyzing, this could be utilized in an automated system for example. Upon a determination that the distal portion of the therapeutic substance delivery device is located or otherwise likely located in the cochlea, the therapeutic substance can be automatically delivered. This can have utilitarian value with respect to a device that is utilized by a non-healthcare professional. In this regard, embodiments include a device and/or system that can be utilized by a person who is in need of therapeutic substance to be delivered to his or her cochlea. The device can be self guiding and/or self inserting, with the recipient holding the device on the outside of the ear braced against the head utilizing a head brace structure, or with the device supported by a structure that lies on a table or on a bed for example, with the human holding his or her head still or having his or her head attached to the device so that general alignment is maintained during the procedure. This arrangement can also be utilized with the robotic system detail below. But briefly, in an exemplary embodiment, there is a system, comprising a therapeutic substance delivery device, such as any of the devices herein (or another type of device, such as a cochlear implant implantation device, or some other implant implantation device). The system further comprises a computational device having the non-transitory medium detailed herein or a medium that has code thereon for executing one or more of the method actions herein (as is the case for all method actions detailed herein—embodiments include a non-transitory computer-readable media having recorded thereon a computer program including code for executing one or more of the method actions detailed herein). The computational device could be the laptop computer detailed above, or a dedicated processor or the like. And also, the compositional device can be a remote processor or a server configured to hold her otherwise store the medium. The connotation of device can also be a smart phone.

In an exemplary embodiment, there is a method that includes the action of automatically executing a therapeutic substance delivery action based on an analysis of the data from the sensors, wherein the therapeutic substance delivery action is an adjustment of an amount of therapeutic substance delivery relative to a prior amount previously delivered. Here, this can encompass using the sensor(s) to monitor e.g. the protein concentration of perilymph over longer times in addition to just when implanted/proximate the time of implantation/access to the inner ear. In an embodiment, the data can be used/is used to adjust therapeutic substance delivery at later time point throughout an implant's life span. This can be analogous to an automated implantable insulin pump that is coupled with a glucose sensor and adjusts insulin dosing based on the glucose level. Accordingly, in an embodiment, the teachings herein are used in conjunction with/part of an implant device that is implanted for a long term period/used days or weeks or months after implantation. In an embodiment, the implant could periodically unshielded electrodes (e.g., unshield one electrode per day or per 2, 3, 4, 5, 6, 7, 8, 9, 10 days, etc.) to obtain data (in an embodiment, once the electrode is unshielded, and for example the coating reacts, it will not be able to be used again, hence multiple electrodes that can be unshielded). In an embodiment, there is use of the implant in a controlled manner when needed. For example, the implant can be controlled to unshield an electrode for testing at a random time (e.g., the human is experiencing some symptom, so the implant is commanded to execute a test at that time). Of course, in some embodiments, the electrodes could be automatically periodically unshielded based on autonomous control of the implant.

In an exemplary embodiment, the system is configured with at least one of an indicator subsystem controlled by the medium for automatically indicating to the healthcare professional the data based on the data (this could be a light (such as one supported by the device of FIG. 8 or sound from a microphone or an indicator on the laptop, etc.) or an automatic dispensing subsystem controlled by the medium for automatically executing the therapeutic substance delivery action. More details of an exemplary system are provided below.

In an exemplary embodiment, the code for analyzing input is code for executing a spectroscopy analysis, and thus in an exemplary method, there is the method action of executing a spectroscopy analysis. In an exemplary embodiment, the spectroscopy of the analysis can be impedance spectroscopy and/or can be dielectric absorption spectroscopy. In an exemplary embodiment, the frequency dependent impedance/absorption spectra have specific peaks/shapes when certain molecules are around or change their concentration. This can be utilized to determine the likelihood of the location of the distal portion of the device and/or to obtain real-time data or semi real time data regarding the state of a physiological future of the person, such as the state of the cochlea or otherwise the state of the perilymph within the cochlea. Embodiments can include the utilization of platinum electrodes in combination with sensitive impedance measurement equipment. In an exemplary embodiment, the devices detailed herein include such sensitive impedance measurement equipment so as to enable the utilized station of spectroscopy analysis to implement at least some of the teachings detailed herein.

In an exemplary embodiment, the frequencies utilized for the impedance spectroscopy can be between 100 Hz to 100 MH or any value or range of values therebetween in 100 Hz increments. Embodiments include devices configured to change the frequency to get a spectrum impedance change, and by obtaining a characteristic impedance spectra, determinations can be made about the substances in contact or otherwise proximate the electrodes. Embodiments thus utilize such determinations and processes to determine a location of a distal portion of the therapeutic substance delivery device by way of example.

In an exemplary embodiment, therapeutic substance include but are not limited to, any of those detailed above, and can include peptides, biologics, cells, drugs, neurotrophics, etc. Any substance that can have therapeutic features if introduced to the cochlea can be utilized.

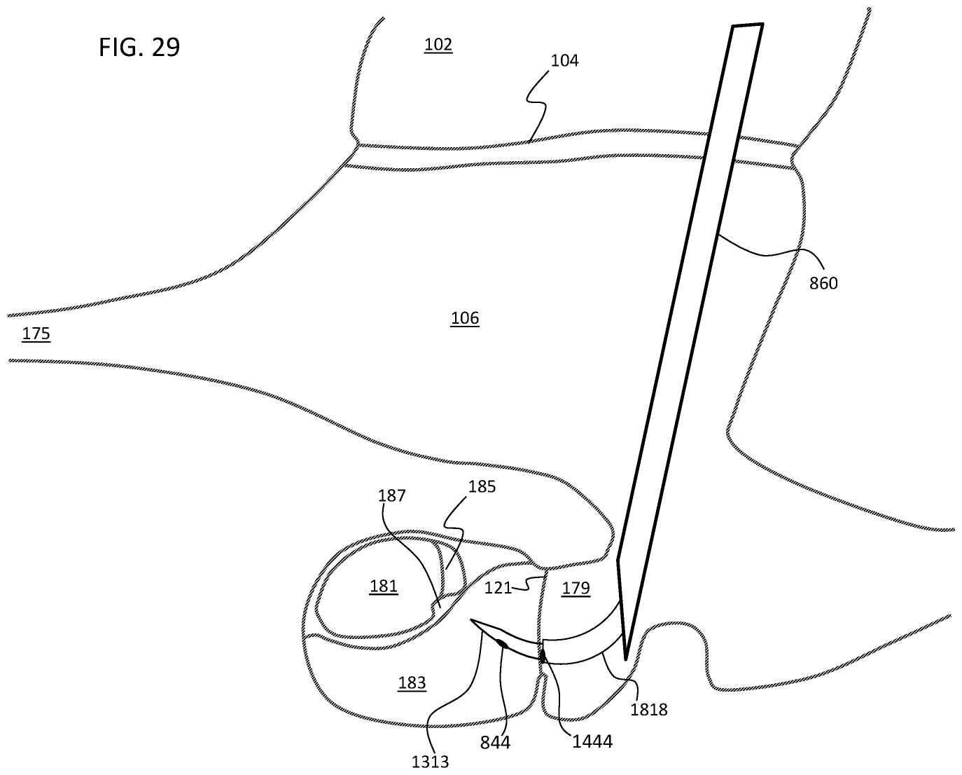

While many of the embodiments above have focused on the placement of two or more electrodes into the cavity at issue, such as a duct of the cochlea, embodiments also include only placing one electrode into the duct. In this regard, FIG. 29 presents an exemplary arrangement where there is an electrode 844 carried by the conduit 1313 that becomes located in the duct 183 of the cochlea. However, there is another electrode 1444 that is located outside the duct, and comes into contact with the round window 121. Here, the idea is that the perilymph and the material of the round window are sufficient to provide a route for electrical current from the electrode 844 that is located inside the cochlea to the electrode 1444 (and vis-a-versa) to implement the teachings detailed herein. Placement of the electrode 1444 can be located elsewhere or otherwise the electrode 1444 need not come into contact with the round window in at least some exemplary embodiments. In an exemplary embodiment, the electrode 1444 can be located at the oval window or otherwise on the bone establishing the boundary between the middle ear and/or the inner ear. Any placement of any sensor component that can enable the teachings detailed herein to have utilitarian value can be utilized in at least some exemplary embodiments.

Some exemplary embodiments include the utilization of devices to implement some or all of the method actions detailed herein. In some exemplary embodiments, these devices include or otherwise are logic circuits or electronics devices, such as processors, which processors canning include or otherwise can have access to memory components. Alternatively, and/or in addition to this, computer chips can be configured or otherwise programmed to execute one or more of the method actions detailed herein. In some embodiments, there is a system, comprising a processor and/or microchip or some form of electronic logic circuitry and a sensor configured to execute at least some of the method actions detailed herein. The logic circuitry can be part of or can be the laptop computer disclosed above or other types of computer devices that can enable the teachings detailed herein that are programmed or otherwise configured to implement at least some of the method actions detailed herein.

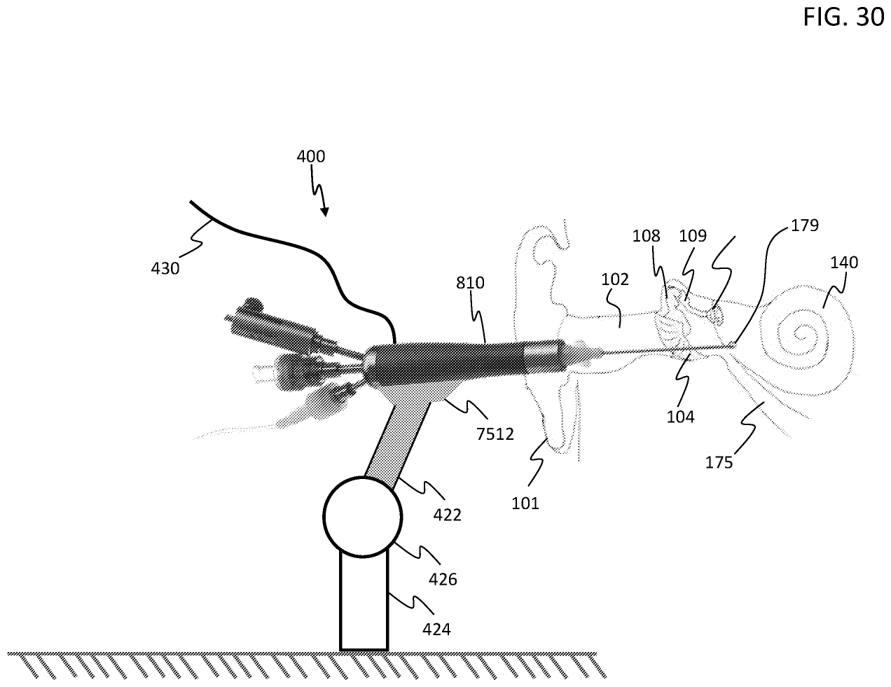

It is noted that at least some exemplary embodiments include utilization of the tools/devices herein and/or variations thereof with a robotic system. In this regard, FIG. 30 is a perspective view of an exemplary embodiment of a therapeutic substance delivery system 400. It is noted that the embodiment depicted in FIG. 30 is presented for conceptual purposes only. Features are provided typically in the singular so as to demonstrate the concept associated therewith. However, it is noted that in some exemplary embodiments, some of these features are duplicated, triplicated, quadplicated, etc. so as to enable the teachings detailed herein and/or variations thereof. Briefly, it is noted that any teaching detailed herein can be combined with a robotic apparatus and/or a robotic system according to the teachings detailed herein and/or variations thereof. In this regard, any method action detailed herein corresponds to a disclosure of a method action executed by a robotic apparatus and/or utilizing a robot to execute that action and/or executing that method action is part of a method where other actions are executed by robot and/or a robotic system etc. Still further, it is noted that any apparatus detailed herein can be utilized in conjunction with a robotic apparatus and/or a robot and/or a system utilizing such. Accordingly, any disclosure herein of an apparatus corresponds to a disclosure of an apparatus that is part of a robotic apparatus and/or a robotic system etc. and/or a system that includes a robotic apparatus etc.

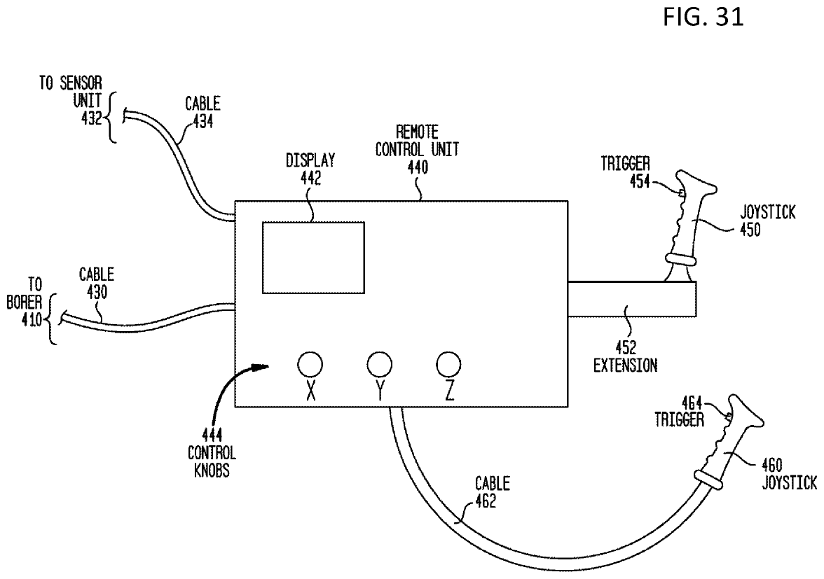

System 400 includes a robotic insertion apparatus having the therapeutic substance delivery device (also referred to as the ear endoscope-again, reference to one is a disclosure of the other, and visa-versa) 810 including a releasable connection to mount 7512, which is supported by a support and movement system, comprising support arm 422 which is connected to joint 426 which in turn is connected to support arm 424. Support arm 424 is rigidly mounted to a wall, a floor, or some other relatively stationary surface. That said, in an alternative embodiment, support arm 424 is mounted to a frame that is attached to the head of the recipient or otherwise connected to the head of the recipient such that global movement of the head will result in no relative movement of the system 400 in general, and the therapeutic substance delivery device in particular, relative to the tissue of the human, such as the cochlea, or tympanic membrane, or round window, or promontory, etc. Joint 426 permits arm 2510, and thus the therapeutic substance delivery device, to be moved in one, two, three, four, five, or six degrees of freedom. (It is noted again that FIG. 31 is but a conceptual FIG.—there can be joints located along the length of various components, such as for example arm 4222 enable that to articulate in the one or more of the aforementioned degrees of freedom at those locations. In an exemplary embodiment, joint 426 includes actuators that move mount 7512, and thus the therapeutic substance delivery device, in an automated manner, as will be described below. In an exemplary embodiment, the system is configured to be remotely controlled via communication with a remote control unit via communication lines of cable 430. In an exemplary embodiment, the system is configured to be automatically controlled via a control unit that is part of the system 400. Additional details of this will be described below.

The system 400 further includes by way of example only and not by way of limitation, sensor/sensing unit 432. That said, in some embodiments, sensor 432 is not part of system 400. In some embodiments, it is a separate system. Still further, in some embodiments, it is not utilized at all with system 400. While sensor 432 is depicted as being co-located simultaneously with the therapeutic substance delivery device, etc., as detailed below, sensor 432 may be used relatively much prior to use of the therapeutic substance delivery device. Sensing unit 432 is configured to scan the head of a recipient and obtain data indicative of spatial locations of internal organs (e.g., mastoid bone 221, middle ear cavity 423 and/or ossicles 106, etc.) In an exemplary embodiment, sensing unit 432 is a unit that is also configured to obtain data indicative of spatial locations of at least some components of the therapeutic substance delivery device and/or other components of the robotic apparatus attached thereto. The obtained data may be communicated to remote control unit 440 via communication lines of cable 434. As may be seen, sensor 432 is mounted to a support and movement system 420 that may be similar to or the same as that used by the robotic apparatus supporting the therapeutic substance delivery device.

In an exemplary embodiment, sensing unit 432 is an MRI system, an X-Ray system, an ultrasound system, a CAT scan system, or any other system which will permit the data indicative of the spatial locations to be determined as detailed herein and/or variations thereof. As will be described below, this data may be obtained prior to surgery and/or during surgery. It is noted that in some embodiments, at least some portions of the therapeutic substance delivery device are configured to be better imaged or otherwise detected by sensing unit 432. In an exemplary embodiment, the tip of the therapeutic substance delivery device includes radio-opaque contrast material. The stop of the therapeutic substance delivery device can also include such radio-opaque contrast material. In an exemplary embodiment, at least some portions of therapeutic substance delivery device in general, and the robotic system in particular, or at least the arm 7510, mount 7512, arm 422, etc., are made of non-ferromagnetic material or other materials that are more compatible with an MRI system or another sensing unit utilized with the embodiment of FIG. 66 than ferromagnetic material or the like. As will be described in greater detail below, the data obtained by sensing unit 432 is used to construct a 3D or 4D model of the recipient's head and/or specific organs of the recipient's head (e.g., temporal bone) and/or portions of the robotic apparatus of which the therapeutic substance delivery device is a part. That said, to be clear, in some embodiments, sensing unit 432 is not present, as seen in FIG. 30.

It is also noted that in some exemplary embodiments of system 400, there are actuators or the like that drive the therapeutic substance delivery device into the ear structure. These actuators can be in signal communication with the control unit. In an exemplary embodiment, the control unit can control the actuators to push the into and/or out of the ear system as will be described in greater detail below. Concomitant with the robotic assembly supporting the therapeutic substance delivery device, in an exemplary embodiment, the control unit is configured to automatically control these actuators.

FIG. 31 is a simplified block diagram of an exemplary embodiment of a remote control unit 440 for controlling the robotic apparatus supporting the therapeutic substance delivery device and sensing unit 432 via communication lines 430 and 434, respectively. Again, it is noted that in some alternate embodiments, the remote control unit 440 is an entirely automated unit. That said, in some alternate embodiments, the remote control unit can be operated automatically as well as manually, which details will be described below.

Remote control unit 440 includes a display 442 that displays a virtual image of the tissue obtained from sensor 432 and/or component(s) of the therapeutic substance delivery device and may superimpose a virtual image of the insertion apparatus onto the virtual image indicative of a current position of the tool relative to the ear anatomy. An operator (e.g., surgeon, certified healthcare provider, etc.) utilizes remote control unit 440 to control some or all aspects of the robotic apparatus and/or sensing unit 432. Exemplary control may include depth of insertion, angle of insertion, speed of advancement and/or retraction of the tool (therapeutic substance delivery device for example), etc. (It is noted that any reference to advancement and/or retraction also corresponds to an alternate disclosure of lateral movement and/or rotation of the tool and/or a change in the angle on any one or more of the three planes relative to the tissue that is the target, etc.) Such control may be exercised via joystick 450 mounted on extension 452 which fixedly mounts joystick 450 to a control unit housing. Such control may be further exercised via joystick 460 which is not rigidly connected to housing of remote control unit 440. Instead, it is freely movable relative thereto and is in communication with the remote control unit via communication lines of cable 462. Joystick 462 may be part of a virtual system in which the remote control unit 440 extrapolates control commands based on how the joystick 462 is moved in space, or joystick may be a device that permits the operator more limited control over the cavity borer 410. Such control may include, for example an emergency stop upon release of trigger 464 and/or directing the robot to drive the therapeutic substance delivery device further into the ear system, etc. by squeezing the trigger 464 (which, in some embodiments, may control a speed at which the therapeutic substance delivery device is advanced by squeezing harder and/or more on the trigger). In the same vein, trigger 454 of joystick 450 may have similar and/or the same functionality.

Control of the robot assembly supporting the therapeutic substance delivery device may also be exercised via knobs 440 which may be used to adjust an angle of the therapeutic substance delivery device in the X, Y and Z axis, respectively. Other controls components may be included in remote control 440.

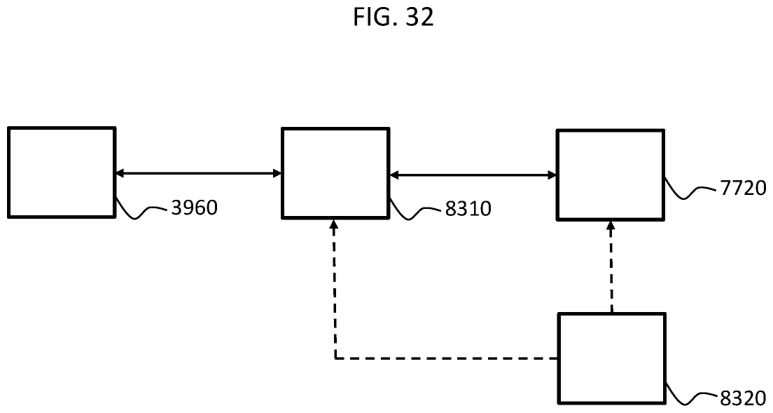

FIG. 32 depicts an exemplary functional schematic of an exemplary system that includes a data collection unit 3960 that receives data from, for example, the sensors of the therapeutic substance delivery device, in signal communication with a control unit 8310 which is in turn in signal communication with an actuator assembly, 7720, where the actuator assembly 7720 is a proxy for a component that positions the therapeutic substance delivery device, or at least advances and/or retracts the therapeutic substance delivery device. The data collection unit and the control unit can be one and the same in some embodiments.

It is also noted that in some embodiments, the there is no control unit. That is, the system can be a purely data collection system, which conveys information to the surgeon or other healthcare professional to instruct (e.g., the output of the control unit and/or the test unit can be instead an instruction as opposed to a control signal) or otherwise provide an indication of the phenomenon to the surgeon or other healthcare professional.

Also functionally depicted in FIG. 32 is the optional embodiment where an input device 8320 is included in the system (e.g., which could be on an embodiment where the actuator assembly 7720 is connected to the therapeutic substance delivery device, but the input device 8320 is located remote from the therapeutic substance delivery device, which could be part of a remote unit 440). In an exemplary embodiment, the input device 8320 could be the trigger 454 and/or 464 of the remote control unit 440. Again, in an exemplary embodiment, the input device 8320 can be utilized to enable advancement and/or withdrawal of the therapeutic substance delivery device and the system 400 could control the advancement and/or withdrawal based on an automated protocol or some other flyby wire type system. In the embodiment of FIG. 32, the input device 8320 can be in signal communication directly to the therapeutic substance delivery device, and/or in signal communication with the control unit 8310.

In an exemplary embodiment, control unit 8310 can correspond to the remote unit 440. That said, in an alternate embodiment, remote unit 440 can be a device that is in signal communication with control unit 8310. Indeed, in an exemplary embodiment, input device 8320 can correspond to remote control unit 440.

More particularly, control unit 8310 can be a signal processor or the like or a personal computer or the like or a mainframe computer or the like etc., that is configured to receive signals from the data collection unit 3960 and analyze those signals to evaluate an insertion status of the therapeutic substance delivery device. More particularly, the control unit 8310 can be configured with software the like to analyze the signals from unit 3960 in real time and/or in near real time as the therapeutic substance delivery device is being advanced by an actuator assembly of the robotic system. The control unit 8310 analyzes the input from test unit 3960 as the therapeutic substance delivery device is advanced by the actuator assembly for example, and evaluates the input to determine if there exists an undesirable insertion status and/or evaluates the input to determine if the input indicates that a scenario could occur or otherwise there exists data in the input that indicates that a scenario is more likely to occur relative to other instances where the insertion status of the therapeutic substance delivery device will become undesirable if the therapeutic substance delivery device is continued to be advanced into the ear system, all other things remaining the same (e.g., insertion angle/trajectory, etc., which can be automatically changed as well via-more on this below). In an exemplary embodiment, upon such a determination, control unit 8310 could halt the advancement of the therapeutic substance delivery device by stopping the actuator(s) of actuator assembly and/or could slow the actuator(s) so as to slow rate of advancement of the therapeutic substance delivery device and/or could reverse the actuator(s) so as to reverse or otherwise retract the therapeutic substance delivery device (either partially or fully). In at least some exemplary embodiments, control unit 8310 can be configured to override the input from input unit 8320 input by the surgeon or the user or the like of the systems herein.

In an exemplary embodiment, the outputs of unit 3960 corresponds to the outputs indicated herein. Alternatively, and/or in addition to this, input into control unit 8310 can flow from other sources. Any input relating to the measurement of voltage associated executing the teachings herein into control unit 8310 can be utilized in at least some exemplary embodiments.

In an exemplary embodiment, control unit 8310 can be configured to determine, based on the input from test unit 3960, whether the therapeutic substance delivery device has come into contact with the tympanic membrane and/or the round window and/or the promontory, etc., and/or that one or more anomalous therapeutic substance delivery device positions has occurred and/or whether there exists an increased likelihood that such will occur, and automatically control the actuator assembly of the insertion system accordingly. In an exemplary embodiment, control unit 8310 does not necessarily determine that such an insertion status exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” the actual insertion status or the forecasted insertion status of the therapeutic substance delivery device, but instead need only be able to control the actuator assembly 7720 based on the input.

In an exemplary embodiment, control unit 8310 can be configured to determine, based on the input from test unit 3960, the insertion depth of the therapeutic substance delivery device and/or a forecasted insertion depth of the therapeutic substance delivery device, and automatically control the actuator assembly 7720 accordingly. In an exemplary embodiment, control unit 8310 does not necessarily determine the insertion depth or forecasted insertion depth, but instead is programmed or otherwise configured so as to control the actuator assembly 7720 according to a predetermined regime based on the input from the test unit 3960. That is, the control unit 8310 need not necessarily “understand” otherwise “know” the actual insertion depth or the forecasted insertion depth of the therapeutic substance delivery device, but instead need only be able to control the actuator assembly 7720 based on the input.

In an exemplary embodiment, control unit 8310 can be configured to determine, based on the input from test unit 3960, executing, for example, the methods/techniques disclosed herein, whether the therapeutic substance delivery device has buckled or has become hung up or has adopted an unutilitarian trajectory and/or position and/or any other anomalous therapeutic substance delivery device location as disclosed herein or otherwise may be the case and/or whether there exists an increased likelihood that such will occur, and automatically control the actuator assembly 7720 accordingly. In an exemplary embodiment, control unit 8310 does not necessarily determine that such, exists or is more likely to exist, but instead is programmed or otherwise configured so as to control the actuator assembly 7720 according to a predetermined regime based on the input from the test unit 3960. That is, the control unit 8310 need not necessarily “understand” otherwise “know” that the condition has occurred or will occur in the future, but instead need only be able to control the actuator assembly 7720 based on the input.

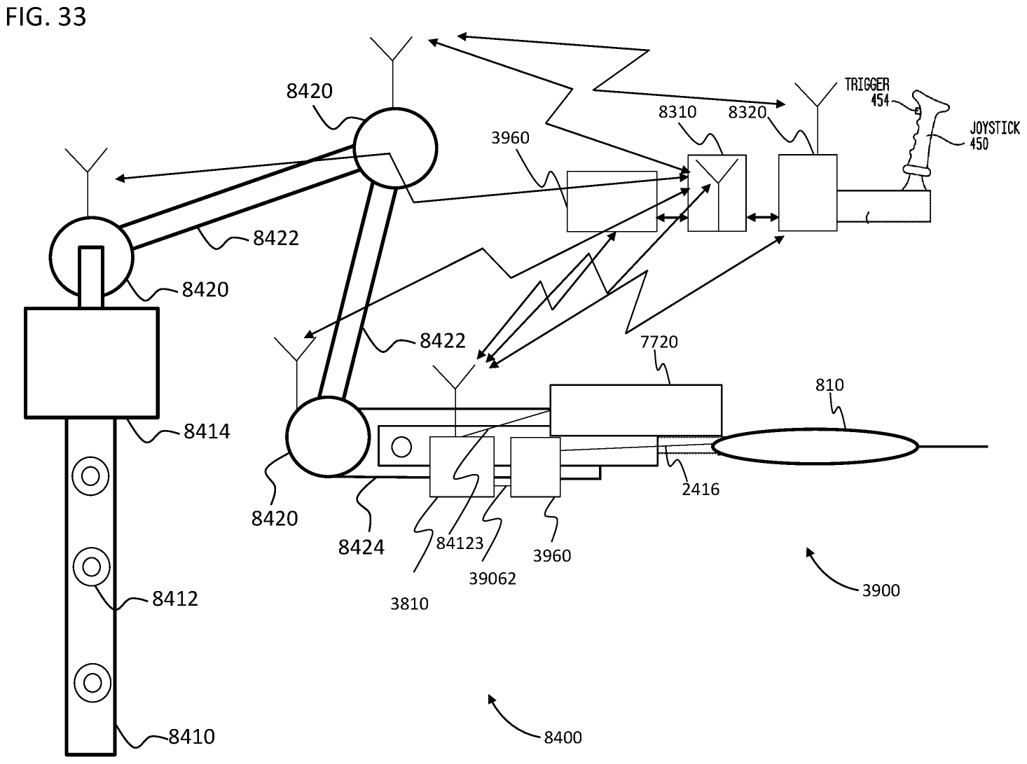

To be clear, while the embodiment detailed above is focused on controlling the actuator assembly 7720 based on data from the system so as to control the advancement and/or retraction of the therapeutic substance delivery device based on the data disclosed herein and, in an alternate embodiment, the system 400 controls one or more other actuators of the robot apparatus of system 400. These one or more other actuators can be exclusive from the actuator assembly 7720, or can include the actuator assembly 7720. In this regard, FIG. 33 depicts an exemplary robot apparatus 8400, that includes the therapeutic substance delivery device detailed above and/or variations thereof with respect to the integration of a system disclosed herein therewith mounted on arm 8424 utilizing bolts in a manner concomitant with that detailed above. In an exemplary embodiment, robot apparatus 8400 has the functionality or otherwise corresponds to that of the embodiment of FIG. 31. In this regard, any functionality associated or otherwise described with respect to the embodiment of FIG. 31 corresponds to that of the embodiment of FIG. 33, and vice versa. In this exemplary embodiment, the actuator apparatus 7720 is in signal communication with unit 3810 via electrical lead 84123. In this regard, signals to and/or from the actuator assembly 7720 can be transmitted to/from the antenna of unit 8310 (the “Y” shaped elements are antennas) and thus communicated via lead 84123. It is briefly noted that while the embodiment depicted in FIG. 33 utilizes radiofrequency communication, in alternate embodiments, the communications can be wired. In an exemplary embodiment both can be utilized.

The robot apparatus 8400 includes a recipient interface 8410 which entails an arch or halo like structure made out of metal or the like that extends about the recipient's cranium or other parts of the body. The interface 8410 is bolted to the recipient's head via bolts 8412. That said, in alternate embodiments, other regimes of attachment can be utilized, such as by way of example only and not by way of limitation, strapping the robot to the recipient's head. In this regard, the body and interface 8410 can be a flexible strapping can be tightened about the recipient's head.

Housing 8414 is located on top of the interface 8410, as can be seen. In an exemplary embodiment, housing 8414 includes a battery or the like or otherwise provides an interface to a commercial/utility power supply so as to power the robot apparatus. Still further, in an exemplary embodiment, housing 8414 can include hydraulic components/connectors to the extent that the actuators herein utilize hydraulics as opposed to and/or in addition to electrical motors. Mounted on housing 8414 is the first actuator 8420, to which arm 8422 is connected in an exemplary embodiment, actuator 8420 enables the components “downstream” (i.e., the arm connected to the actuator, and the other components to the therapeutic substance delivery device) to articulate in one, two, three, four, five or six degrees of freedom. A second actuator 8420 is attached to the opposite end of the arm 8422, to which is attached a second arm 8422, to which is attached a third actuator 8420, to which is attached to the therapeutic substance delivery device attachment structure 8424. Elements 8422 and 8424 can be metal beams, such as I beams or C beams or box beams, etc. actuators 8420 can be electrical actuators and/or hydraulic actuators.

As can be seen, each actuator 8420 is provided with an antenna, which antenna is in signal communication with the control unit 8310. In an exemplary embodiment, control unit 8310 can control the actuation of those actuators 8420 so as to position the therapeutic substance delivery device 3900 (generically identified-reference numeral 810 is also used) at the desired position relative to the recipient. That said, in an alternate embodiment, a single antenna can be utilized, such as one mounted on housing 8414, which in turn is connected to a decoding device that outputs a control signal, such as a driver signal based on the decoded RF signal, to the actuators 8420 (as opposed to each actuator having such a device), which control signals can be provided via a wired system/electrical leads extending from housing 8414 to the actuators. Note also that in some alternate embodiments, control unit 8310 is in wired communication with the actuators, either directly or indirectly, and/or is in wired communication with the decoding device located in the housing 8414. Any arrangement that can enable control of the robot apparatus in general, and the actuators thereof in particular, via control unit 8310 can be utilized in at least some exemplary embodiments.

Note also that while the embodiment depicted in FIG. 33 is such that the actuators 8420 must actuate so as to extend the therapeutic substance delivery device into the body, in an alternate embodiment, as noted above, the therapeutic substance delivery device can be mounted on a rail system or the like, wherein a cylindrical actuator or the like pushes the therapeutic substance delivery device in a linear manner into the head and withdrawals the therapeutic substance delivery device in the linear manner from the head. In an exemplary embodiment, this actuator apparatus can enable one degree of freedom movements of the therapeutic substance delivery device, while in other embodiments, this actuator apparatus can enable two or three or four or five or six degrees of freedom. Indeed, in an exemplary embodiment, this actuator apparatus can enable movement only in a linear direction, but can enable rotation of the therapeutic substance delivery device about the longitudinal axis thereof. Any arrangement of actuator assemblies that will enable the therapeutic substance delivery device to be positioned relative to the ear system via robotic positioning thereof can be utilized in at least some exemplary embodiments.

Any control unit and/or test unit or the like disclosed herein can be a personal computer programs was to execute one or more or all of the functionalities associated there with are the other functionalities disclosed herein. In an exemplary embodiment, any control unit and/or test unit or the like can be a dedicated circuit assembly configured so as to execute one or more or all of the functionalities associated there with or the other functionalities disclosed therein. In an exemplary embodiment, and the control unit and/or test unit or the like disclosed herein can be a processor or the like or otherwise can be a programmed processor.

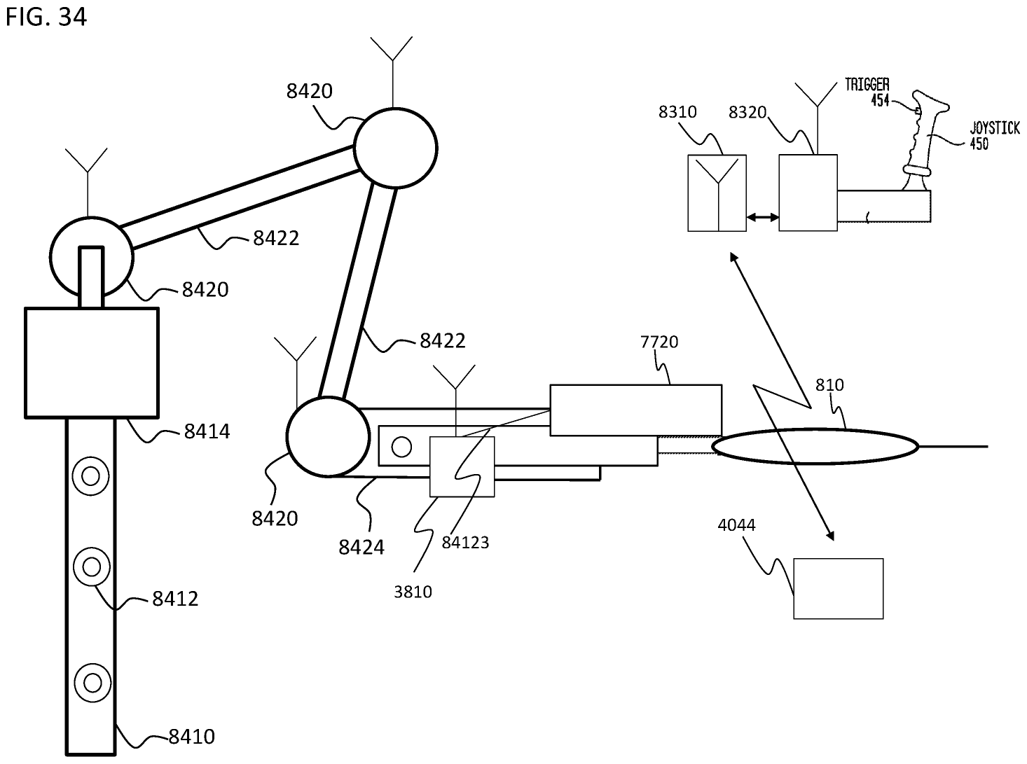

FIG. 34 depicts another exemplary embodiment, as seen. FIG. 34 presents such an exemplary embodiment, with the links between the antennas removed for clarity. Testing system 4044 detailed shown in signal communication with control unit 8310. In this exemplary embodiment, system 4044 corresponds to that detailed above vis-à-vis determining anomalous therapeutic substance delivery device location with the exception that it is entirely divorced from the therapeutic substance delivery device, save for the communication between system 4044 and the control unit 8310, to the extent such is relevant for the purposes of discussion, where control unit 8310 is in signal communication with one or more of the assemblies of the robot apparatus, such as the actuator assembly 7720. Here, during insertion, and/or prior to insertion and/or after insertion, the system 4044 monitors or otherwise measures phenomenon detailed herein and communicates those measurements and/or the analysis thereof to control unit 8310, which analyzes those signals and develops a control regime for therapeutic substance delivery device insertion and/or therapeutic substance delivery device positioning based on those signals. Note also that in some exemplary embodiments, the system 4044 can have multiple measurement sensors, some of which are part of the robot apparatus, and some of which are separate from the robot apparatus, all of which are part of system 4044. Alternatively, these various components of the system 4044 can communicate with test unit 3960. Such can have utilitarian value with respect to a scenario where measurements are first taken prior to placing the therapeutic substance delivery device near the pertinent tissues and after inserting the therapeutic substance delivery device into the ear system, where it is undesirable to have the therapeutic substance delivery device proximate certain tissue. Any device, system, and/or method that will enable controlled movement of the therapeutic substance delivery device relative to the ear system and/or cochlea based on phenomenon associated with the recipient can be utilized in at least some exemplary embodiments.

Again, the test unit and the system 4044 can be one and the same in some embodiments, and in some embodiments, functionality can be bifurcated between the two as separate units. Indeed, element 4044 in FIG. 34 can be a proxy for the control unit and/or the test units detailed above.

It is briefly noted that any reference to a therapeutic substance delivery device and/or a drill bit and/or a hand tool herein corresponds to a disclosure of an alternate embodiment that includes that feature in a more generic tool and/or in another of the tools disclosed herein, and visa-versa.

It is noted that any disclosure of a device and/or system herein corresponds to a disclosure of a method of utilizing such device and/or system. It is further noted that any disclosure of a device and/or system herein corresponds to a disclosure of a method of manufacturing such device and/or system. It is further noted that any disclosure of a method action detailed herein corresponds to a disclosure of a device and/or system for executing that method action/a device and/or system having such functionality corresponding to the method action. It is also noted that any disclosure of a functionality of a device herein corresponds to a method including a method action corresponding to such functionality. Also, any disclosure of any manufacturing methods detailed herein corresponds to a disclosure of a device and/or system resulting from such manufacturing methods and/or a disclosure of a method of utilizing the resulting device and/or system.

Embodiments include embodiments where any or more of the teachings detailed herein are combined with any one or more of the other teachings detailed herein, unless otherwise noted, providing that the art enables such. Embodiments also include embodiments where any one or more of the teachings detailed herein are specifically excluded from combination with any one or more of the other teachings detailed herein unless otherwise noted providing that the art enables such.

Unless otherwise specified or otherwise not enabled by the art, any one or more teachings detailed herein with respect to one embodiment can be combined with one or more teachings of any other teaching detailed herein with respect to other embodiments, and this includes the duplication or repetition of any given teaching of one component with any like component. It is also noted that embodiments can include devices systems and/or methods that specifically exclude one or more of the disclosures presented herein (i.e., it is not present).

While various embodiments of the present invention have been described above, it should be understood that they have been presented by way of example only, and not limitation. It will be apparent to persons skilled in the relevant art that various changes in form and detail can be made therein without departing from the scope of the invention.

1. A method, comprising:
entering a fluid containing cavity in a human with an artificial device; and
verifying that a portion of the artificial device has entered the fluid containing cavity based on an electrical phenomenon indicative of a sensor component supported by and/or part of the artificial device being located in the fluid containing cavity.

2. The method of claim 1, wherein:
the electrical phenomenon is based on impedance.

3. The method of claim 1, wherein:
the electrical phenomenon is a change in a value indicative of the electrical phenomenon.

4. (canceled)

5. The method of claim 1, wherein:
the sensor component is an electrode.

6. The method of claim 1, wherein:
the sensor component is shielded immediately prior to entering the fluid containing cavity to reduce a statistical likelihood that the electrical phenomenon will be experienced before the sensor component becomes located in the fluid containing cavity.

7. The method of claim 1, wherein:
the fluid filled cavity is a cochlea of a human.

8. (canceled)

9. The method of claim 1, wherein:
the action of verifying that the portion of the artificial device is based on an increase in electrical conductivity between the sensor component and another component due to the sensor component coming into contact with perilymph.

10-22. (canceled)

23. A non-transitory computer-readable media having recorded thereon, a computer program for executing at least a portion of a method, the computer program including:
code for analyzing input from a sensor indicative of electrical phenomena within a cochlea; and
code for at least one of:
automatically indicating to a healthcare professional data based on data indicative of a sensor component being located in a cochlea based on an analysis executed using the code for analyzing; or
automatically executing a therapeutic substance delivery action based on an analysis executed using the code for analyzing.

24. The medium of claim 23, wherein:
the code for analyzing input is code for executing a spectroscopy analysis.

25. The medium of claim 24, wherein:
the spectroscopy is impedance spectroscopy.

26. (canceled)

27. The medium of claim 23, wherein:
the medium includes the code for automatically indicating to a healthcare professional data based on data indicative of a sensor component being located in a cochlea based on an analysis executed using the code for analyzing.

28. The medium of claim 23, wherein:
the medium includes the code for automatically executing a therapeutic substance delivery action based on an analysis executed using the code for analyzing.

29. A system, comprising:
a therapeutic substance delivery device; and
a computational device having the non-transitory medium of claim 23, wherein
the system is configured with at least one of:
an indicator subsystem controlled by the medium for automatically indicating to the healthcare professional the data based on the data; or
an automatic dispensing subsystem controlled by the medium for automatically executing the therapeutic substance delivery action.

30. The medium of claim 23, wherein:
the medium includes the code for automatically executing a therapeutic substance delivery action based on an analysis executed using the code for analyzing, wherein the therapeutic substance delivery action is an adjustment of an amount of therapeutic substance delivery relative to a prior amount previously delivered.

31. A method, comprising:
obtaining access to a middle ear of a human;
moving a device sized and dimensioned to fit at least partially into a duct of a cochlea in a direction believed to be towards a duct of a cochlea from the middle ear; and
artificially verifying that a distal portion of the device is statistically likely to be located in the cochlea.

32. The method of claim 31, wherein:
the device is a therapeutic substance delivery device; and
the therapeutic substance delivery device carries sensor components;
the sensor components are used to obtain data indicative of location and/or lack of location of the distal portion in the duct of the cochlea; and
the obtained data is used to execute the action of artificially verifying.

33. The method of claim 31, wherein:
the device is a drill bit; and
the drill bit carries sensor components;
the sensor components are used to obtain data indicative of location and/or lack of location of the distal portion in the duct of the cochlea; and
the obtained data is used at least as part of the action of executing the action of artificially verifying.

34. The method of claim 31, further comprising:
providing a fluid and/or a semi-fluid substance into the middle ear cavity, the substance having a known property;
sensing a feature of the known property during a first time period; and
ceasing to sense the feature of the known property during a second time period following the first time period, wherein
the action of ceasing to sense the feature is used at least as part of the action of executing the action of artificially verifying.

35. The method of claim 31, wherein:
the device carries and/or includes a substance that chemically reacts with another substance in the duct of the cochlea;
the method includes having the substance chemically react with the another substance in the cochlea and obtaining data indicative of the chemical reaction having taken place; and
the action of obtaining data indicative of the chemical reaction is used at least as part of the action of artificially verifying.

36. The method of claim 31, wherein:
the device carries and/or includes a structure that accepts and/or accretes a substance located in the duct of the cochlea;
the method includes having the substance accepted by the structure and/or having the substance accreted by the structure and obtaining data indicative of the acceptance and/or accretion having taken place; and
the action of obtaining data is used at least as part of the action of artificially verifying.

37. The method of claim 31, further comprising:
delivering a therapeutic substance into the duct of the cochlea after artificially verifying that the distal portion of the device is statistically likely to be located in the cochlea.

38-40. (canceled)
